SEQUENCE LISTING (18 sequences)

SEQ ID NO: 1 - DNA, 27 bp, Human Immunodeficiency Virus
gccacctttgtcgactgttaaaaaact

SEQ ID NO: 2 - DNA, 24 bp, Human Immunodeficiency Virus
gtcctaggcaagcttcctggatgc

SEQ ID NO: 3 - DNA, 25 bp, Human Immunodeficiency Virus
aaggagacggatgggtgcgagagcg

SEQ ID NO: 4 - DNA, 26 bp, Human Immunodeficiency Virus
ggggatccctttattgtgacgagggg

SEQ ID NO: 5 - DNA, 25 bp, Human Immunodeficiency Virus
attgtgggccatgggcgcgagaaac

SEQ ID NO: 6 - DNA, 24 bp, Human Immunodeficiency Virus
ggggggcccctactggtcttttcc

SEQ ID NO: 7 - DNA, 28 bp, Human Immunodeficiency Virus
gaagatctaccatggaagccccagaaga

SEQ ID NO: 8 - DNA, 39 bp, Human Immunodeficiency Virus
cgcggatccgttaacatctactggctccatttcttgctc

SEQ ID NO: 9 - DNA, 25 bp, Human Immunodeficiency Virus
gtgcaacaccatggcaggccccaga

SEQ ID NO: 10 - DNA, 39 bp, Human Immunodeficiency Virus
tgcactgcaggaagatcttagacctggagggggaggagg

SEQ ID NO: 11 - DNA, 17 bp, Human Immunodeficiency Virus
agtagatgttgggatcc

SEQ ID NO: 12 - protein, 5 aa, Human Immunodeficiency Virus
SRCWD

SEQ ID NO: 13 - DNA, 29 bp, Human Immunodeficiency Virus
ctaagatcggggagctcactagtggatcc

SEQ ID NO: 14 - protein, 9 aa, Human Immunodeficiency Virus
LRSGSSLVD

SEQ ID NO: 15 - DNA, 21 bp, Human Immunodeficiency Virus
agtagatgttgggatctaatg

SEQ ID NO: 16 - protein, 7 aa, Human Immunodeficiency Virus
SRCWDLM

SEQ ID NO: 17 - DNA, 33 bp, Human Immunodeficiency Virus
ctaagatcggggagctcactagtggatctaatg

SEQ ID NO: 18 - protein, 11 aa, Human Immunodeficiency Virus
LRSGSSLVDLM

(19) United States
(12) Patent Application Publication
(10) Pub. No.: US 2003/0157690 A1
(43) Pub. Date: Aug. 21, 2003

(54) Fusion protein delivery system and uses thereof

(75) Inventors: John C. Kappes, Birmingham, AL (US); Xiaoyun Wu, Birmingham, AL (US)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: UAB Research Foundation

(21) Appl. No.: 10/306,885

(22) Filed: Nov. 27, 2002

Related U.S. Application Data
(63) Continuation of application No. 09/578,786, filed on May 23, 2000, now abandoned.
Application No. 09/578,786 is a continuation of application No. 09/089,900, filed on Jun. 3, 1998, now abandoned.
(63) Continuation-in-part of application No. 08/947,516, filed on Sep. 29, 1997, now Pat. No. 6,001,985.
Application No. 08/947,516 is a continuation of application No. 08/421,982, filed on Apr. 14, 1995, now abandoned.

(51) Int. Cl. C12N 15/867; C07H 21/04; C12N 7/00

(52) U.S. Cl. 435/235.1; 435/456; 435/320.1; 536/23.72

(57) ABSTRACT

The present invention provides a composition of matter, comprising: DNA encoding a viral Vpx protein fused to DNA encoding a protein. In another embodiment of the present invention, there is provided a composition of matter, comprising: DNA encoding a viral Vpr protein fused to DNA encoding a protein. The present invention further provides DNA, vectors and methods for expressing a lentiviral pol gene in trans, independent of the lentiviral gag-pol. A gene transduction element is optionally delivered to a lentiviral vector according to the present invention. Also provided are various methods of delivering a virus inhibitory molecule to a target in an animal. Further provided is a pharmaceutical composition.

### RELATED APPLICATION

This patent application is a continuation-in-part of patent application Ser. No. 08/947,516 filed Sep. 29, 1997, which is a file-wrapper continuation of patent application Ser. No. 08/421,982, both prior applications also being entitled “Fusion Protein Delivery System and Uses Thereof.”

### BACKGROUND OF THE INVENTION

1. Field of the Invention

The present invention relates generally to the fields of molecular virology and protein chemistry. More specifically, the present invention relates to the use of Human and Simian Immunodeficiency Virus (HIV/SIV) Vpx and Vpr proteins, or amino acid residues that mediate their packaging, as vehicles for delivery of proteins/peptides to virions or virus-like particles and uses thereof.

2. Description of the Related Art

Unlike simple retroviruses, human and simian immunodeficiency viruses (HIV/SIV) encode proteins in addition to Gag, Pol, and Env that are packaged into virus particles. These include the Vpr protein, present in all primate lentiviruses, and the Vpx protein, which is unique to the HIV-2/SIV_{SM}/SIV_{MAC} group of viruses. Since Vpr and Vpx are present in infectious virions, they have long been thought to play important roles early in the virus life cycle. Indeed, recent studies of HIV-1 have shown that Vpr has nucleophilic properties and that it facilitates, together with the matrix protein, nuclear transport of the viral preintegration complex in nondividing cells, such as the macrophage. Similarly, Vpx-deficient HIV-2 has been shown to exhibit delayed replication kinetics and to require 2-3 orders of magnitude more virus to produce and maintain a productive infection in peripheral blood mononuclear cells. Thus, both accessory proteins appear to be important for efficient replication and spread of HIV/SIV in primary target cells.

Incorporation of foreign proteins into retrovirus particles has previously been reported by fusion with gag. Using the yeast retrotransposon Tyl as a retrovirus assembly model, Natsoulis and Boeke tested this approach as a novel means to interfere with viral replication. More recently, the expression of a murine retrovirus capsid-staphylococcal nuclease fusion protein was found to inhibit murine leukemia virus replication in tissue culture cells.

Lentiviral vectors, specifically those based on HIV-1, HIV-2 and SIV, have utility in gene therapy, due to their attractive property of stable integration into nondividing cell types (15, 25, 34). The utility of lentiviral-based vector use for human therapy requires the development of a safe lentiviral-based vector. HIV virion associated accessory proteins (Vpr and Vpx) have been shown to be useful as vehicles to deliver protein of both viral and non-viral origin into HIV particles (11, 12, 28-30). We recently demonstrated that trans-RT and IN mimics cis-RT and IN (derived from Gag-Pol). The trans-RT and IN proteins effectively rescue the infectivity and replication of virions derived from RT-IN minus provirus through the complete life cycle (12, 28). Moreover, these findings demonstrate that truncated Gag-Pol precursor polyprotein (Gag-Pro) support the formation of infectious particles when the functions of RT and IN are provided in trans. This finding demonstrated for the first time for a lentivirus that the full length Gag-Pol precursor is not required for the formation of infectious particles. Our data also show that trans Vpr-RT-IN, or Vpr-RT together with Vpr-IN are fully functional and support virus infectivity, integration of the proviral DNA, and replication (through one cycle) of RT defective, IN defective and RT-IN defective viruses (ref. 12 and 28). It should also be noted that our data demonstrate that enzymatically active RT does not require Vpr for incorporation into virions (FIGS. 19A and B). RT can be incorporated into HIV-1 virions when expressed in trans even without its expression as a fusion partner of Vpr. These data demonstrate that the functions of these critical enzymes can be provided in trans, independent of their normal mechanism for expression and virion incorporation as components of the Gag-Pol precursor protein.

The prior art is deficient in the lack of effective means of delivering or targeting foreign, e.g., toxic proteins to virions. The present invention fulfills this longstanding need and desire in the art.

### SUMMARY OF THE INVENTION

Vpr and Vpx packaging is mediated by the Gag precursor and thus must play an important role in HIV assembly processes. The present invention shows that Vpr and Vpx can be used as vehicles to target proteins of viral and non-viral origin into HIV/SIV virions. Vpr1 and Vpx2 gene fusions were constructed with bacterial staphylococcal nuclease (SN) and chloramphenicol acetyl transferase (CAT) genes. Unlike Gag or Pol proteins, Vpr and Vpx are dispensable for viral replication in immortalized T-cell lines. Thus, structural alteration of these accessory proteins may be more readily tolerated than similar changes in Gag or Gag/Pol. Fusion proteins containing a Vpx or Vpr moiety should be packaged into HIV particles by expression in trans, since their incorporation should be mediated by the same interactions with Gag that facilitates wild-type Vpr and Vpx protein packaging.

Vpr and Vpx fusion proteins were constructed and their abilities to package into HIV particles were demonstrated. Fusion partners selected for demonstration were: staphylococcal nuclease because of its potential to degrade viral nucleic acid upon packaging and the chloramphenicol acetyl transferase because of its utility as a functional marker. To control for cytotoxicity, an enzymatically inactive nuclease mutant (SN*), derived from SN by site-directed mutagenesis was also used. This SN* mutant differs from wild-type SN by two amino acid substitutions; Glu was changed to Ser (position 43) and Arg was changed to Gly (position 87). SN* folds normally, but has a specific activity that is 10⁶-fold lower than wild-type SN. Using transient expression systems and in trans complementation approaches, fusion protein stability, function and packaging requirements were shown. The present invention shows that Vpr1 and Vpx2 fusion proteins were expressed in mammalian cells and were incorporated into HIV particles even in the presence of wild-type Vpr and/or Vpx proteins. More importantly, however, the present invention shows that virion incorporated Vpr and Vpx fusions remain enzymatically active. Thus, targeting heterologous Vpr and Vpx fusion proteins, including deleterious enzymes, to virions represents a new avenue toward anti-HIV drug discovery. For example, utilizing Vpr as a delivery vehicle to incorporate an HIV-1/SIV protease mutant (enzymatically defective, D25N) into wild type HIV-2 and SIV particles, we found that the PR-mutant interfered with normal viral proteolytic processing and virion maturation, which resulted in a defect in the infectivity of the wt virus (ref. 29). These results show that we can target HIV PR and PR mutants into the HIV particle by expression trans, as fusion partners of Vpr and Vpx.

The invention shows that virion associated accessory proteins (Vpr) are operative as vehicles to deliver fully functional RT and IN into HIV particles, independently of their normal expression as components of the Gag-Pol precursor protein; and that infectious particle formation can be achieved by expressing GagPro, when RT and IN functions are provided in trans. Therefore this invention generates a novel packaging component (Gag-Pro), and a novel trans-enzymatic element that provides enzyme function for lentiviral-based vectors. The present invention affords a safer antiviral vector, in part by diminishing the likelihood of generating replication competent retrovirus through genetic recombination. The packaging system of the present invention provides RT and IN separate from the Gag and Gag-Pol precursor, by the expression of RT and IN in trans as fusion partners of Vpr. The generation of recombinants is therefore decreased relative to the prior art systems. According to the present invention, the generation of potentially infectious/replicating retroviral forms (LTR-gag-pol-LTR) is decreased, since in our approach this requires recombination of three separate RNAs derived from the different plasmids: transduction plasmid, packaging plasmid and RT-IN expression plasmid, and as such is unlikely to occur. Virion associated accessory proteins (Vpr and by analogy Vpx) are utilized in the present invention as vehicles to deliver the RT and IN proteins into lentiviral vectors, independently of Gag and Gag-Pol. As such, a “trans-lentiviral” vector is utilized for gene delivery, and gene therapy.

In one embodiment of the present invention, there is provided a composition of matter, comprising: DNA encoding a viral Vpx protein fused to DNA encoding a virus inhibitory protein.

In another embodiment of the present invention, there is provided a composition of matter, comprising: DNA encoding a viral Vpr protein fused to DNA encoding a virus inhibitory protein.

In yet another embodiment of the present invention, there is provided a method of delivering a virus inhibitory molecule to a target in an animal, comprising the step of administering to said animal an effective amount of tile composition of the present invention.

In still yet another embodiment of the present invention, there is provided a pharmaceutical composition, comprising a composition of the present invention and a pharmaceutically acceptable carrier.

Other and further aspects, features, and advantages of the present invention will be apparent from the following description of the presently preferred embodiments of the invention given for the purpose of disclosure.

### BRIEF DESCRIPTION OF THE DRAWINGS

So that the matter in which the above-recited features, advantages and objects of the invention, as well as others which will become clear, are attained and can be understood in detail, more particular descriptions of the invention briefly summarized above may be had by reference to certain embodiments thereof which are illustrated in the appended drawings. These drawings form a part of the specification. It is to be noted, however, that the appended drawings illustrate preferred embodiments of the invention and therefore are not to be considered limiting in their scope.

FIG. 1 shows the construction of vpr1, vpr1SN/SN*, vpx2 and vpx2SN/SN* expression plasmids. FIG. 1A shows the illustration of the pTM-vpr1 expression plasmid. The HIV-1_{YU2} vpr coding region was amplified by PCR and ligated into pTM1 at the NcoI and BamHI restriction sites. FIG. 1B shows the illustration of the pTM-vpx2 expression plasmid. The HIV-2_{ST} vpx coding region was amplified by PCR and ligated into pTM1 at the NcoI and Bgl II/SmaI sites. FIG. 1C shows the illustration of the fusion junctions of the pTM-vpr1SN/SN* expression plasmids. SmaI/XhoI DNA fragments containing SN and SN* were ligated into HpaI/XhoI cut pTM-vpr1. Blunt-end ligation at HpaI and SmaI sites changes the vpr translational stop codon (TAA) to Trp and substituted the C terminal Ser with a Cys residue. FIG. 1D shows the illustration of the fusion junctions of the pTM-vpx2SN/SN* expression plasmids. BamHI/XhoI DNA fragments containing SN and SN* were ligated into BamHI/XhoI cut pTMvpx2. In the construction of these plasmids, the Vpx C terminal Arg codon was changed to a Val codon and a Ser residue was introduced in place of the Vpx translational stop codon (TAA). Fusion of vpx and SN/SN* at the BamHI sites left a short amino acid sequence of the pTM1 polylinker (double underlined) between the two coding regions.

FIG. 2 shows the expression of Vpr1- and VPX2- SN and SN* fusion proteins in mammalian cells. FIG. 2A shows the pTM1, pTM-vpr1, pTM-vpr1SN and pTM-vpr1SN* were transfected into HeLa cells one hour after infection with rVT7 (MOI=10). Twenty-four hours later cell lysates were prepared and examined by immunoblot analysis. Replica blots were probed with anti-Vpr1 (left) and anti-SN (right) antibodies. FIG. 2B shows that replica blots, prepared from rVT7 infected HeLa cells transfected with pTM1, pTM-vpx2, pTM-vpx2SN and pTM-vpx2SN*, were probed with anti-Vpx2 (left) and anti-SN (right) antibodies. Bound antibodies were detected by ECL (Amersham) methods as described by the manufacturer.

FIG. 3 shows the incorporation of Vpr1- and Vpx2- SN and SN* fusion proteins into virus-like particles (VLP). FIG. 3A transfection of T7 expressing (rVT7 infected) HeLa cells with pTM-vpr1, pTM-vpr1 SN, and pTM-vpr1 SN* alone and in combination with pTM-gag1. pTM1 was also transfected for control. Culture supernatant were collected twenty-four hours after transfection, clarified by centrifugation (1000×g, 10 min.) and ultracentrifuged (125,000×g, 2 hrs.) over cushions of 20% sucrose. Pellets (VLPs, middle and bottom panels) and cells (top panel) were solubilized in loading buffer and examined by immunoblot analysis using anti-Vpr1 (top and middle) and anti-Gag (bottom) antibodies as probes. FIG. 3B transfection of T7 expressing HeLa cells pTM-vpx2, pTM-vpx2SN and pTM-vpx2SN* alone and in combination with pTM-gag2. Pellets (VLPs, middle and bottom panels) and cells (top panel) were lysed, proteins were separated by SDS-PAGE and electroblotted blotted to nitrocellulose as described above. Replica blots were probed with anti-Vpx2 (top and middle panels) and anti-Gag (bottom panel) antibodies. Bound antibodies were detected using ECL methods.

FIG. 4 shows that virus-specific signals mediate incorporation of Vpr and Vpx- SN into VLPs. FIG. 4A shows that HIV-1 Gag mediates packaging of Vpr1SN. rVT7 infected (T7 expressing) HeLa cells were transfected with pTM-vpr1SN alone and in combination with pTM-gag2 and pTM-gag1. Pellets (VLPs, middle and bottom panels) and cells (top panel) were prepared 24 hours after transfection and examined by immunoblot analysis using anti-Vpr1 (top and middle) and anti-Gag (bottom) antibodies for probes. (B) HIV-2 Gag mediates packaging of Vpx2SN. T7 expressing HeLa cells were transfected with pTM-vpx2SN alone and in combination with pTM-gag I and pTM-gag2. Pellets (VLPs, middle and bottom panels) and cells (top panel) were prepared 24 hours after transfection and examined by immunoblot analysis using anti-Vpx2 (top and middle) and anti-Gag (bottom) antibodies for probes.

FIG. 5 shows a competition analysis of Vpr1SN and Vpx2SN for incorporation into VLPs. FIG. 5A shows transfection of T7 expressing HeLa cells with different amounts of pTM-vpr1 (2.5, 5 and 10 ug) and pTM-vpr1SN (2.5, 5 and 10 ug), either individually or together in combination with pTM-gag I (10 ug). FIG. 5B shows that HeLa cells were transfected with different amounts of pTM-vpx2 (2.5, 5 and 10 ug) and pTM-vpx2SN (2.5, 5 and 10 ug), either individually or together with pTM-gag2 (10 ug). Twenty hours after transfection, particles were concentrated by ultracentrifugation through sucrose cushions and analyzed by immunoblotting using anti-Vpr1 (A) or anti-Vpx2 (B) antibodies.

FIG. 6 shows the nuclease activity of VLP-associated Vpr1SN and Vpx2SN proteins. Virus-like particles were concentrated from culture supernatants of T7 expressing HeLa cells cotransfected with pTM-gag1/pTM-vpr1SN, pTM-gag1/pTM-vpr1SN*, pTM-gag2/pTM-vpx2SN and pTM-gag2/pTM-vpx2SN* by ultracentrifugation (125,000×g, 2 hrs.) through 20% cushions of sucrose. Pellets containing Vpr1-SN and SN* (B) and Vpx2-SN and SN* (C) were resuspended in PBS. Tenfold dilutions were made in nuclease reaction cocktail buffer (100 mM Tris-HCl pH 8.8, 10 mM CaCl₂, 0.1% NP40) and boiled for 1 minute. 5 ul of each dilution was added to 14 ul of reaction cocktail buffer containing 500 ng of lambda phage DNA (HindIII fragments) and incubated at 37° C. for 2 hours. Reaction products were electrophoresed on 0.8% agarose gels and DNA was visualized by ethidium bromide staining. Standards (A) were prepared by dilution of purified staphylococcal nuclease (provided by A. Mildvan) into cocktail buffer and assayed.

FIG. 7 shows the incorporation of Vpx2SN into HIV-2 by trans complementation. FIG. 7A shows the construction of the pLR2P-vpx2SN/SN* expression plasmids. To facilitate efficient expression of HIV genes, the HIV-2 LTR and RRE were engineered into the polylinker of pTZ19U, generating pLR2P. The organization of these elements within the pTZ19U polylinker is illustrated. NcoI/XhoI vpx2SN and vpx2SN* (vpxSN/SN*) containing DNA fragments were ligated into pLR2P, generating pLR2P-vpx2SN and pLR2P-vpx2SN* (pLR2P-vpxSN/SN*). FIG. 7B shows the association of Vpx2SN with HIV-2 virions. Monolayer cultures of HLtat cells were transfected with HIV-2_{ST} proviral DNA (pSXB1) and cotransfected with pSYB1/pTM-vpx2SN and pSXBI/pTM-vpx2SN*. Extracellular virus was concentrated from culture supernatants forty-eight hours after transfection by ultracentrifugation (125,000×g, 2 hrs.) through cushions of 20% sucrose. Duplicate Western blots of viral pellets were prepared and probed independently with anti-Vpx2 (left) anti-SN (middle) and anti-Gag (right) antibodies. FIG. 7C shows a sucrose gradient analysis. Pellets of supernatant-virus prepared from pSXB1/pTM-vpx2SN cotransfected HLtat cells were resuspended in PBS, layered over a 20-60% linear gradient of sucrose and centrifuged for 18 hours at 125,000×g. Fractions (0.5 ml) were collected from the bottom of the tube, diluted 1:3 in PBS, reprecipitated and solubilized in electrophoresis buffer for immunoblot analysis. Replica blots were probed with anti-SN (top) and anti-Gag (bottom) antibodies. Fraction 1 represents the first collection from the bottom of the gradient and fraction 19 represents the last collection. Only alternate fractions are shown, except at the peak of protein detection. FIG. 7D shows the incorporation of Vpx2SN into HIV-2_{7312A} Vpr and Vpx competent virus. Virus concentrated from supernatants of HLtat cells transfected with HIV-2_{7312A} proviral DNA (pJK) or cotransfected with PJK/pLR2P-vpx2SN or pJK/pLR2P-vpx2SN* was prepared for immunoblot analysis as described above. Included for control were virions derived by pSXB1/pLR2P-vpx2SN* cotransfection. Duplicate blots were probed with anti-Vpx (left) and anti-Gag (right) antibodies.

FIG. 8 shows the incorporation of Vpr1SN into HIV-1 virions by trans complementation. Culture supernatant virus from HLtat cells transfected with pNL4-3 (HIV-1) and pNL4-3R (HIV-1 vpr mutant) or cotransfected with pNL4-3/pLR2P-vpr1SN and pNL4-3R/pLR2P-vpr1SN was prepared for immunoblot analysis as described above. Blots were probed with anti-SN (FIG. 8A), anti-Vpr1 (FIG. 8B) and anti-Gag (FIG. 8C) antibodies.

FIG. 9 shows the inhibition of Vpr1/Vpx2-SN processing by an HIV protease inhibitor. HIV-1 (pSG3) and HIV-2 (pSXB1) proviral DNAs were cotransfected separately into replica cultures of HLtat cells with pLR2P-vpr1SN and pLR2P-vpx2SN, respectively. One culture of each transfection contained medium supplemented with 1 uM of the HIV protease inhibitor L-699-502. Virions were concentrated from culture supernatants by ultracentrifugation through cushions of 20% sucrose and examined by immunoblot analysis using anti-Gag (FIG. 9A) and anti-SN (FIG. 9B) antibodies.

FIG. 10 shows the incorporation of enzymatically active Vpr1- and Vpx2-CAT fusion proteins into HIV virions. FIG. 10A shows an illustration of the fusion junctions of the pLR2P-vpr1 CAT and pLR2P-vpx2CAT expression plasmids. PCR amplified BamHI/XhoI DNA fragments containing CAT were ligated into BglII/XoI cut pLR2P-vpr1SN and pLR2P-vpx2SAN, replacing SN (see FIG. 1). This construction introduced two additional amino acid residues (Asp and Leu, above blackened bar) between the vpr1/vpx2CAT coding regions.

FIG. 10B shows the incorporation of Vpr1CAT into HIV-1 virions. Virus produced from HLtat cells transfected with pNL4-3 (HIV-1) and pNL4-3R (HIV1-R), or cotransfected with pNL4-3/pLR2P-vpr1CAT and pNL4-3R-/pLR2Pvpr1CAT was prepared as described above and examined by immunoblot analysis. Replica blots were probed with anti-Vpr1 (left) and anti-Gag (right) antibodies. FIG. 10C shows the incorporation of Vpx2CAT into HIV-2 virions. Virus produced from HLtat cells transfected with pSXB1 (HIV-2) or cotransfected with pSXB1/pLR2P-vpx2CAT was prepared as described above and examined by immunoblot analysis. Replica blots were probed with anti-Vpx2 (left) and antiGag (right) antibodies. FIG. 10D shows that virion incorporated Vpr1- and Vpx2-CAT fusion proteins possess enzymatic activity. Viruses pelleted from HLtat cells transfected with pSXB1 (HIV-2) or cotransfected with pSXB1/pLR2P-vpx2CAT and pNL4-3/pLR2P-vpr1CAT were lysed and analyzed for CAT activity. HIV-2 was included as a negative control.

FIG. 11 shows virion association of enzymatically active CAT and SN fusion proteins. FIG. 11A shows that HIV-2 virions collected from the culture supernatant of HLtat cells cotransfected with pSXB1 and pLR2P-vpx2 were sedimented in linear gradients of 20-60% sucrose. 0.7 ml fractions were collected and analyzed by immunoblot analysis using Gag monoclonal antibodies as a probe. FIG. 11B shows CAT enzyme activity was determined in each fraction by standard methods. The positions of nonacetylated [¹⁴C]chloramphenicol (Cm) and acetylated chloramphenicol (Ac-Cm) are indicated. FIG. 11C shows HIV-1 virions derived from HLtat cells cotransfected with pSG3 and pLR2P-vpr1SN and cultured in the presence of L-689,502 were sedimented in linear gradients of 20-60% sucrose. Fractions were collected and analyzed for virus content by immunoblot analysis using Gag monoclonal antibodies. FIG. 11D shows that SN activity was determined in each fraction as described in FIG. 6.

FIG. 12 shows the HIV-1 genome, the construction of pΔ8.2, pCMV-VSV-G, pHR-CMV-β-gal, pCR-gag-pro, pLR2P-vpr-RT-IN, pCMV-VSV-G and pHR-CMV-β-gal plasmids. FIG. 12A shows an illustration of the HIV-1 genome. FIG. 12B shows the lentivirus vector plasmid expression system. FIG. 12C shows the illustration of a trans-lentiviral vector expression system, where RT and IN are contiguous as Vpr fusion partners.

FIG. 13 shows positive gene transduction with a trans-lentiviral vector of the instant invention as determined by fluorescence microscopy.

FIG. 14 shows positive gene transduction with a lentiviral vector as a control as determined by fluorescence microscopy.

FIG. 15 shows the construction of a pHR-CFTR trans-lentiviral vector of the present invention.

FIG. 16 shows the expression of CFTR on HeLa cells using the trans-lentiviral vector, and the lentiviral vector as a control. Transduced cells were probed with polyclonal antibodies in immunofluorescence microscopy.

FIG. 17 shows the expression of CFTR on HeLa cells using monoclonal antibodies in immunofluorescence microscopy.

FIG. 18 shows the restoration of CFTR function in trans-lentiviral transduced HeLa cells as measured by a halide sensitive fluorophore.

FIGS. 19A and B show the presence in progeny virions of RT in trans without Vpr-dependent incorporation.

FIG. 20 shows that both Vpr-RT and RT support vector transduction when provided in trans.

### DETAILED DESCRIPTION OF THE INVENTION

As used herein, the term “fusion protein” refers to either the entire native protein amino acid sequence of Vpx (of any HIV-2 and SIV) and Vpr (of any HIV-1 and SIV) or any subtraction of their sequences that have been joined through recombinant DNA technology and are capable of association with either native HIV/SIV virions or virus like particles.

As used herein, the term “virion” refers to HIV-1, HIV-2 and SIV virus particles.

As used herein, the term “virus-like particle” refers to any composition of HIV-1, HIV-2 and SIV proteins other than which exists naturally in naturally infected individuals or monkey species that are capable of assembly and release from either natural or immortalized cells that express these proteins.

As used herein, the term “transfect” refers to the introduction of nucleic acids (either DNA or RNA) into eukaryotic or prokaryotic cells or organisms.

As used herein, the term “gene transduction element” refers to the minimal required genetic information to transduce a cell with a gene.

As used herein, the term “virus-inhibitory protein” refers to any sequence of amino acids that have been fused with Vpx or Vpr sequences that may alter in any way the ability of HIV-1, HIV-2 or SIV viruses to multiply and spread in either individual cells (prokaryotic and eukaryotic) or in higher organisms. Such inhibitory molecules may include: HIV/SIV proteins or sequences, including those that may possess enzymatic activity (examples may include the HIV/SIV protease, integrase, reverse transcriptase, Vif, Nef and Gag proteins) HIV/SIV proteins or proteins/peptide sequences that have been modified by genetic engineering technologies in order to alter in any way their normal function or enzymatic activity and/or specificity (examples may include mutations of the HIV/SIV protease, integrase, reverse transcriptase, Vif, Nef and Gag proteins), or any other non viral protein that, when expressed as a fusion protein with Vpr or Vpx, alter virus multiplication and spread in vitro or in vivo.

In the present invention, the HIV Vpr and Vpx proteins were packaged into virions through virus type-specific interactions with the Gag polyprotein precursor. HIV-1 Vpr (Vpr1) and HIV-2 Vpx (Vpx2) are utilized to target foreign proteins to the HIV particle as their open reading frames were fused in-frame with genes encoding the bacterial staphylococcal nuclease (SN), an enzymatically inactive mutant of SN (SN*), and the chloramphenicol acetyl transferase (CAT). Transient expression in a T7-based vaccinia virus system demonstrated the synthesis of appropriately sized Vpr1SN/SN* and Vpx2SN/SN* fusion proteins which, when co-expressed with their cognate p55^{Gag} protein, were efficiently incorporated into virus-like particles (VLPs). Packaging of the fusion proteins was dependent on virus type-specific determinants, as previously seen with wild-type Vpr and Vpx proteins. Particle associated Vpr1SN and Vpx2SN fusion proteins were enzymatically active as determined by in vitro digestion of lambda phage DNA. To demonstrate that functional Vpr1 and Vpx2 fusion proteins were targeted to HIV particles, the gene-fusions were cloned into an HIV-2 LTR/RRE regulated expression vector and co-transfected with wild-type HIV-1 and HIV-2 proviruses. Western blot analysis of sucrose gradient purified virions revealed that both Vpr1 and Vpx2 fusion proteins were efficiently packaged regardless of whether SN, SN* or CAT were used as C terminal fusion partners. Moreover, the fusion proteins remained enzymatically active and were packaged in the presence of wild-type Vpr and Vpx proteins. Interestingly, virions also contained smaller sized proteins that reacted with antibodies specific for the accessory proteins as well as SN and CAT fusion partners. Since similar proteins were absent from Gag-derived VLPs as well as in virions propagated in the presence of an HIV protease inhibitor, they must represent cleavage products produced by the viral protease. Taken together, these results demonstrate that Vpr and Vpx can be used to target functional proteins, including potentially deleterious enzymes, to the HIV/SIV particle. These properties are useful for the development of novel antiviral strategies.

The present invention provides for a delivery of a trans protein or gene to a viral vector through coupling to either a viral protein or gene delivery, respectively; wherein the viral protein is Vpr or Vpx and the gene encodes either Vpr or Vpx. Certain truncations of these trans protein or genes perform the regulatory or enzymatic functions of the full sequence protein or gene. For example, the nucleic acid sequences coding for protease, integrase, reverse transcriptase, Vif, Nef, Gag, RT, IN and CFTR can be altered by substitutions, additions, deletions or multimeric expression that provide for functionally equivalent proteins or genes. Due to the degeneracy of nucleic acid coding sequences, other sequences which encode substantially the same amino acid sequences as those of the naturally occurring proteins may be used in the practice of the present invention. These include, but are not limited to, nucleic acid sequences comprising all or portions of the nucleic acid sequences encoding all above proteins, which are altered by the substitution of different codons that encode a functionally equivalent amino acid residues within the sequence, thus producing a silent change. For example, one or more amino acid residues within a sequence can be substituted by another amino acid of a similar polarity which acts as a functional equivalent, resulting in a silent alteration. Substitutes for an amino acid within the sequence may be selected from other members of the class to which the amino acid belongs. For example, the nonpolar (hydrophobic) amino acids include alanine, leucine, isoleucine, valine, proline, phenylalanine, tryptophan and methionine. The polar neutral amino acids include glycine, serine, threonine, cysteine, tyrosine, asparagine, and glutamine. The positively charged (basic) amino acids include arginine, lysine and histidine. The negatively charged (acidic) amino acids include aspartic acid and glutamic acid. Also included within the scope of the present invention are proteins or fragments or derivatives thereof which are differentially modified during or after translation, e.g., by glycosolation, protolytic cleavage, linkage to an antibody molecule or other cellular ligands, etc. In addition, the recombinant ligand encoding nucleic acid sequences of the present invention may be engineered so as to modify processing or expression of a ligand. For example, a signal sequence may be inserted upstream of a ligand encoding sequence to permit secretion of the ligand and thereby facilitate apoptosis.

Additionally, a ligand encoding nucleic acid sequence can be mutated in vitro or in vivo to create and/or destroy translation, initiation, and/or termination sequences or to create variations in coding regions and/or form new restriction endonuclease sites or destroy pre-existing ones, to facilitate further in vitro modification. Any technique for mutagenesis known in the art can be used, including but not limited to in vitro site directed mutagenesis, J. Biol. Chem 253:6551, use of Tab linkers (Pharmacea), etc.

The following examples are given for the purpose of illustrating various embodiments of the invention and are not meant to limit the present invention in any fashion.

### EXAMPLE 1

### Cells and Viruses

HeLa, HeLa-tat (HLtat) and CV-1 cells were maintained in Dulbecco's Modified Eagle's Medium supplemented with 10% fetal bovine serum (FBS). HLtat cells constitutively express the first exon of HIV-1 tat and were provided by Drs. B. Felber and G. Pavlakis. A recombinant vaccinia virus (rVT7) containing the bacteriophage T7 RNA polymerase gene was used to facilitate expression of viral genes placed under the control of a T7 promoter. Stocks of rVT7 were prepared and titrated in CV-1 cells as described previously by Wu, et al., J. Virol, 66:7104-7112 (1992). HIV-1_{YU2}, HIV-1 pNL 4-3-R and pNL 4-3, HIV-1_{HXB2D}, HIV-2_{ST}, and HIV-2_{7312A} proviral clones were used for the construction of recombinant expression plasmids and the generation of transfection derived viruses.

### EXAMPLE 2

### Antibodies

To generate HIV-1 Vpr specific antibodies, the HIV-1_{YU-2} vpr open reading frame was amplified by polymerase chain reaction (PCR) using primers (sense: 5′GCCACCTTTGTCGACTGTTAAAAAACT-3′ (Seq. Id. No. 1) and anti-sense: 5′-GTCCTAGGCAAGCTTCCTGGATGC-3′) (Seq. Id. No. 2) containing SalI and HindIII sites and ligated into the prokaryotic expression vector, pGEX, generating pGEX-vpr1. This construct allowed expression of Vpr1 as a C terminal fusion protein and glutathione S-transferase (gst), thus allowing protein purification using affinity chromatography. E. coli (DH5a) were transformed with pGEX-vpr1 and protein expression was induced with isopropyl β-D thiogalactopyranoside (IPTG). Expression of the gst-Vpr1 fusion protein was confirmed by SDS-PAGE. Soluble gst-Vpr1 protein was purified and Vpr1 was released by thrombin cleavage using previously described procedures of Smith, et al., Gene 67:31-40 (1988). New Zealand White rabbits were immunized with 0.4 mg of purified Vpr1 protein emulsified 1:1 in Freunds complete adjuvant, boosted three times at two week intervals with 0.25 mg of Vpr1 mixed 1:1 in Freunds' incomplete adjuvant and bled eight and ten weeks after the first immunization to collect antisera. Additional antibodies used included monoclonal antibodies to HIV-1 Gag (ACT1, and HIV-2 Gag (6D2.6), polyclonal rabbit antibodies raised against the HIV-2 Vpx protein and anti-SN antiserum raised against purified bacterially expressed SN protein.

### EXAMPLE 3

### Construction of T7-Based Expression Plasmids

A DNA fragment encompassing ^{HIV-1}HXB2D^{gag} (nucleotides 335-1837) was amplified by PCR using primers (sense: 5′-AAGGAGAGCCATGGGTGCGAGAGCG-3′ (Seq. Id. No. 3) and anti-sense: 5′GGGGATCCCTTTATTGTGACGAGGGG-3′) (Seq. Id. No. 4) containing NcoI and BamHI restriction sites (underlined). The PCR product was digested with NcoI and BamHI, purified and ligated into the polylinker of the pTM1 vector, generating pTM-gag1. Similarly, a DNA fragment containing the gag coding region of HIV-2_{ST} (nucleotides 547-2113) was amplified by PCR using sense and anti-sense primers 5′-ATTGTGGGCCATGGCGCGAGAAAC-3′ (Seq. Id. No. 5) and 5′GGGGGGCCCCTACTGGTCTTTTCC-3′ (Seq. Id. No. 6), respectively. The reaction product was cut with NcoI and SmaI (underlined), purified and ligated into the polylinker of pTM1, generating pTM-gag2.

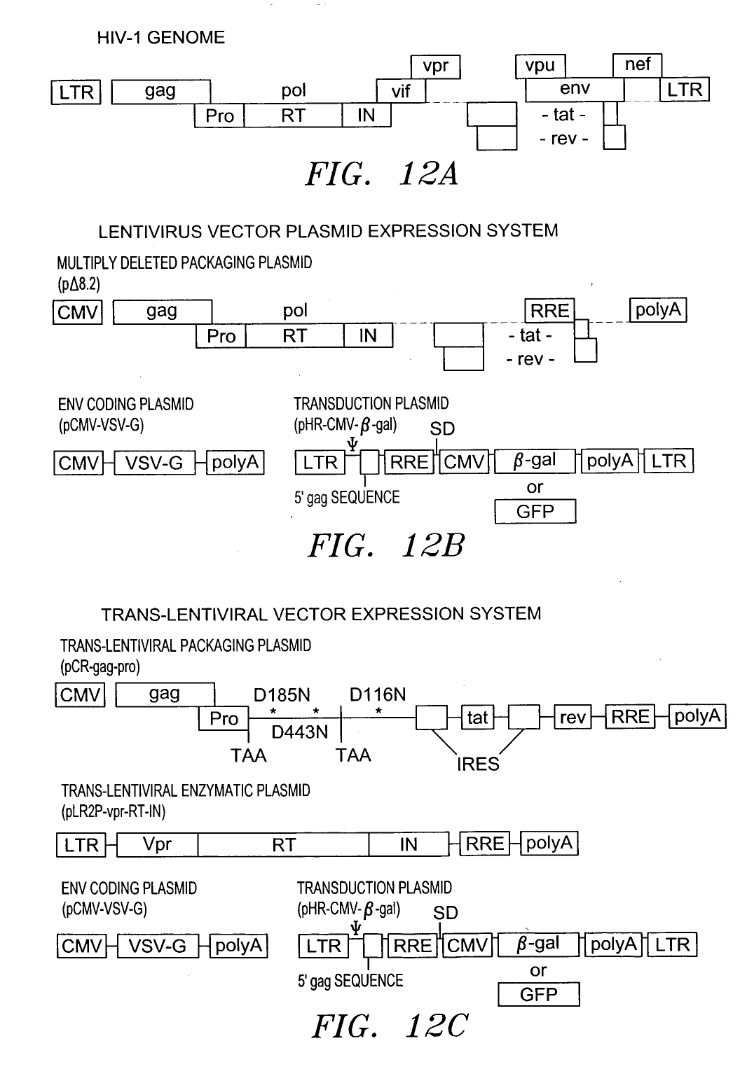

For expression of Vpr1 under the control of the T7 promoter, a DNA fragment containing the HIV-1_{YU2} vpr coding region (nucleotides 5107-5400) was amplified by PCR using primers (sense: 5′GAAGATCTACCATGGAAGCCCCAGAAGA-3′ (Seq. Id. No. 7) and anti-sense: 5′-CGCGGATCCGTTAACATCTACTGGCTCCATTTCTTGCTC-3′) (Seq. Id. No. 8) containing NcoI and HpaI/BamHI sites, respectively (underlined). The reaction product was cut with NcoI and BamHI and ligated into pTM1, generating a pTM-vpr1 (FIG. 12A). In order to fuse SN and SN* in-frame with vpr1, their coding regions were excised from pGN1561.1 and pGN1709.3, respectively and through a series of subcloning steps, ligated into the SmaI/XhoI sites of pTM-vpr1, generating pTM-vpr1 SN and pTM-vpr1 SN*. This approach changed the translational stop codon of Vpr1 to a Trp codon and the C terminal Ser residue to a Cys. The resulting junctions between vpr1 and SN/SN* are depicted in FIG. 12C.

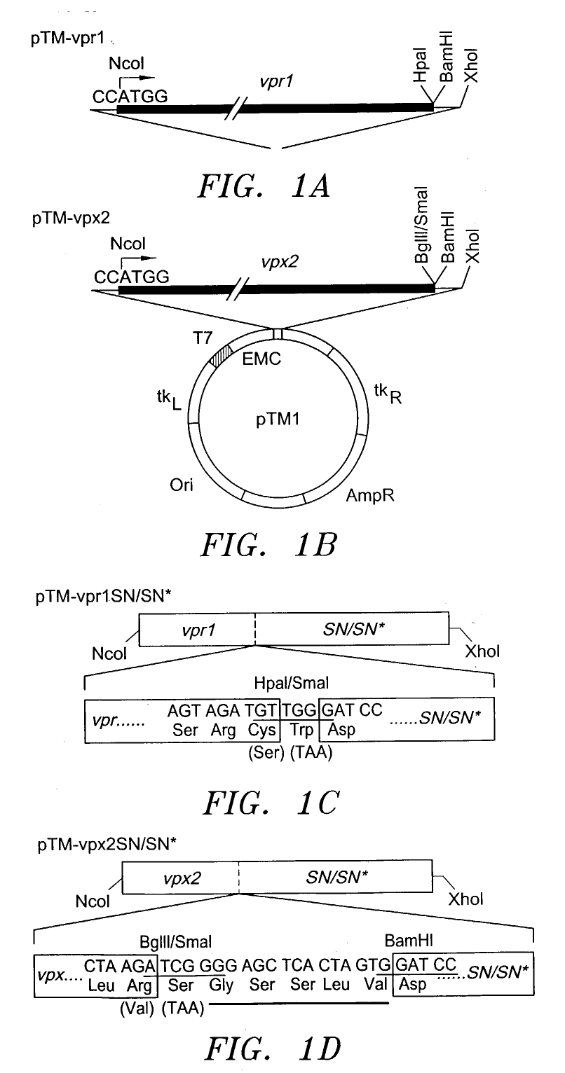

For expression of Vpx2 under T7 control, a DNA fragment containing the HIV-2_{ST} vpx coding sequence (nucleotides 5343-5691) was amplified by PCR using primers (sense: 5′GTGCAACACCATGGCAGGCCCCAGA-3′ (Seq. Id. No. 9) and antisense: 5′-TGCACTGCAGGAAGATCTTAGACCTGGAGGGGGAGGAGG-3′ (Seq. Id. No. 10)) containing NcoI and BglII sites, respectively (underlined). After cleave with BglII and Klenow fill-in, the PCR product was cleaved with NcoI, purified and ligated into the NcoI and SmaI sites of pTM1, generating pTM-vpx2 (FIG. 12B). To construct in-frame fusions with vpx2, BamHI/XhoI, SN- and SN*-containing DNA fragments were excised from pTM-vpr1SN and pTM-vpr1SN* and ligated into pTM-vpx2, generating pTM-vpx2SN and pTM-vpx2SN*, respectively. This approach introduced one amino acid substitution at the C terminus of Vpx (Val to Arg), changed the translational stop codon of vpx to Ser and left five amino acids residues of the pTM1 plasmid polylinker. The resulting junctions between vpx2 and SN/SN* are depicted in FIG. 1D.

### EXAMPLE 4

### Construction of HIV LTR-Based Expression Plasmids

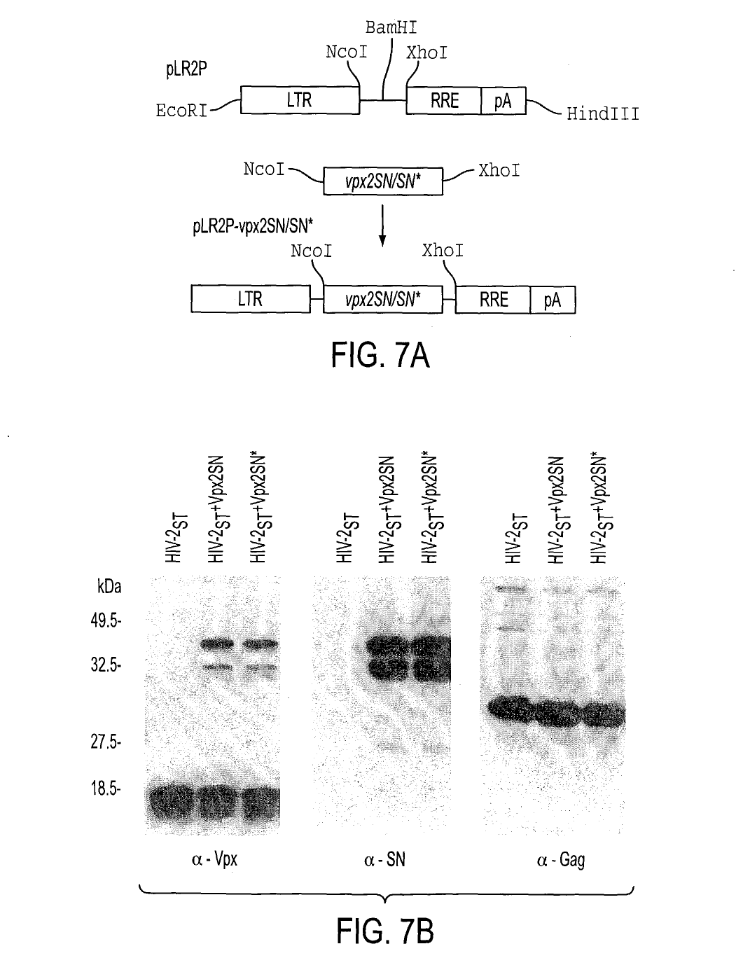

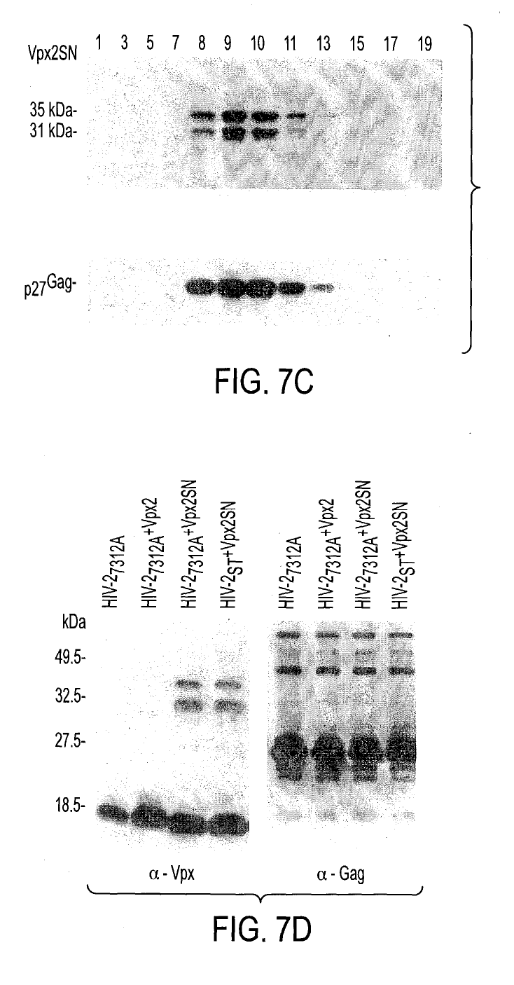

For efficient expression of Vpr and Vpx fusion proteins in the presence of HIV, a eukaryotic expression vector (termed pLR2P) was constructed which contains both an HIV-2 LTR (HIV-2_{ST}, coordinates −544 to 466) and an HIV-2 RRE (HIV-2_{ROD}, coordinates 7320 to 7972) element (FIG. 7A). These HIV-2 LTR and RRE elements were chosen because they respond to both HIV-1 and HIV-2 Tat and Rev proteins. The vpr1, vpr1SN, vpx2 and vpx2SN coding regions were excised from their respective pTM expression plasmids (see FIG. 1) with NcoI and XhoI restriction enzymes and ligated into pLR2P, generating pLR2P-vpr1, pLR2P-vpr1SN, pLR2P-vpx2 and pLR2P-vpx2SN, respectively (FIG. 7A). For construction and expression of vpr- and vpx-CAT gene fusions, the SN containing regions (BamHI/XhoI fragments) of pLR2P-vpr1SN and pLR2P-vpx2SN were removed and substituted with a PCR amplified BglII/Xhol DNA fragment containing CAT, generating pLR2P-vpr1CAT and pLR2P-vpx2CAT, respectively (FIG. 9A).

### EXAMPLE 5

### Transfections

Transfections of proviral clones were performed in HLtat cells using calcium phosphate DNA precipitation methods as described by the manufacturer (Strategene). T7-based (pTM1) expression constructs were transfected using Lipofectin (BioRad) into rVT7 infected HeLa cells as described previously by Wu, et al., J. Virol, 68:6161-6169 (1994). These methods were those recommended by the manufacturer of the Lipofectin reagent.

### EXAMPLE 6

### Western Immunoblot Analysis

Virions and virus-like particles (VLPs) were concentrated from the supernatants of transfected or infected cells by ultracentrifugation through 20% cushions of sucrose (125,000×g, 2 hrs., 4° C.). Pellets and infected/transfected cells were solubilized in loading buffer [62.5 mM Tris-HCl (pH 6.8) 0.2% sodium dodecyl sulfate (SDS), 5% 2-mercaptoethanol, 10% glycerol], boiled and separated on 12.5% polyacrylamide gels containing SDS. Following electrophoresis, proteins were transferred to nitrocellulose (0.2 μm; Schleicher & Schuell) by electroblotting, incubated for one hour at room temperature in blocking buffer (5% nonfat dry milk in phosphate buffered saline [PBS]) and then for two hours with the appropriate antibodies diluted in blocking buffer. Protein bound antibodies were detected with HRP-conjugated specific secondary antibodies using ECL methods according to the manufacturer's instructions (Amersham).

### EXAMPLE 7

### SN Nuclease Activity Assay

Cells and viral pellets were resuspended in nuclease lysis buffer (40 mM Tris-HCl, pH 6.8, 100 mM NaCl, 0.1% SDS, 1% Triton X-100) and clarified by low speed centrifugation (1000×g, 10 min.). Tenfold dilutions were made in nuclease reaction cocktail buffer (100 mM Tris-HCl, pH 8.8, 10 mM CaCl₂, 0.1% NP40) and boiled for 1 minute. 5 μl of each dilution was added to 14 μl of reaction cocktail buffer containing 500 ng of lambda phage DNA (HindIII fragments) and incubated at 37° C. for 2 hours. Reaction products were electrophoresed on 0.8% agarose gels and DNA was visualized by ethidium bromide staining.

### EXAMPLE 8

### Expression of Vpr1 and Vpx2-SN and SN* Fusion Proteins in Mammalian Cells

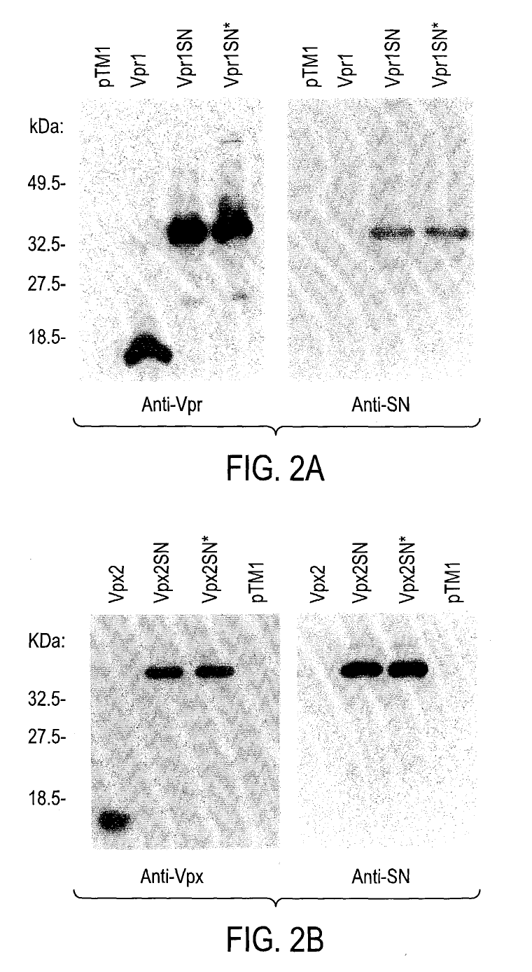

Expression of Vpr1- and Vpx2- SN/SN* fusion proteins in mammalian cells was assessed using the recombinant vaccinia virus-T7 system (rVT7). HeLa cells were grown to 75-80% confluency and transfected with the recombinant plasmids pTM-vpr, pTM-vpx, pTM-vpr1SN/SN*, and pTM-vpx2SN/SN* (FIG. 1). Twenty-four hours after transfection, cells were washed twice with PBS and lysed. Soluble proteins were separated by SDS-PAGE and subjected to immunoblot blot analysis. The results are shown in FIG. 2. Transfection of pTM-vpr1SN and pTM-vpr1SN* resulted in the expression of a 34 kDa fusion protein that was detectable using both anti-Vpr and anti-SN antibodies (A). Similarly, transfection of pTM-vpx2SN and pTM-vpx2SN* resulted in the expression of a 35 kDa fusion protein which was detected using anti-Vpx and antiSN antibodies (B). Both fusion proteins were found to migrate slightly slower than expected, based on the combined molecular weights of Vpr1 (14.5 kDa) and SN (16 kDa) and Vpx2 (15 kDa) and SN, respectively. Transfection of pTM-vpr1 and pTM-vpx2 alone yielded appropriately sized wild-type Vpr and Vpx proteins. Anti-Vpr, anti-Vpx and anti-SN antibodies were not reactive with lysates of pTM1 transfected cells included as controls. Thus, both SN and SN* fusion proteins can be expressed in mammalian cells.

### EXAMPLE 9

### Incorporation of Vpr1- and Vpr2- SN/SN* Fusion Proteins into Virus-Like Particles

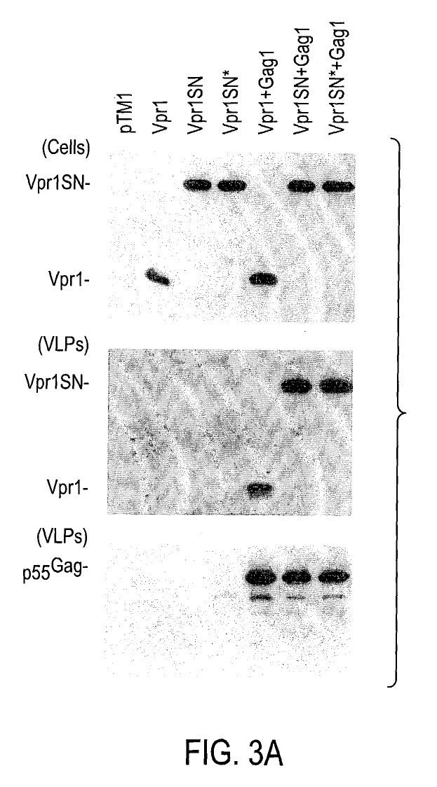

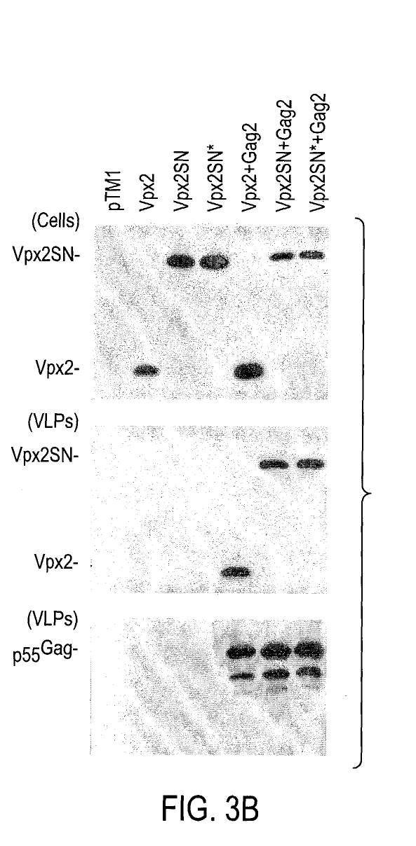

In vaccinia and baculovirus systems, the expression of HIV Gag is sufficient for assembly and extracellular release of VLPs. Vpr1 and Vpx2 can be efficiently incorporated into Gag particles without the expression of other viral gene products. To demonstrate that the Vpri and Vpx2 fusion proteins could be packaged into VLPs, recombinant plasmids were coexpressed with HIV-1 and HIV-2 Gag proteins in the rVT7 system. pTM-vpr1, pTM-vpr1SN and pTM-vpr1 SN* were transfected into HeLa cells alone and in combination with the HIVI Gag expression plasmid, pTM-gag1. Twenty-four hours after transfection, cell and VLP extracts were prepared and analyzed by immunoblot analysis (FIG. 3A). Anti-Vpr antibody detected Vpr1, Vpr1 SN and Vpr1 SN* in cell lysates (top panel) and in pelleted VLPs derived by coexpression with pTM-gag1 (middle panel). In the absence of HIV-1Gag expression, Vpr1 and Vpr1SN were not detected in pellets of culture supernatants (middle panel). As expected VLPs also contained p55 Gag (bottom panel). Thus, Vpr1SN/SN* fusion proteins were successfully packaged into VLPs.

To demonstrate that Vpx2SN was similarly capable of packaging into HIV-2 VLPs, pTM-vpx2, pTM-vpx2SN and pTM-vpx2SN* were transfected into HeLa cells alone and in combination with the HIV-2 Gag expression plasmid, pTM-gag2. Western blots were prepared with lysates of cells and VLPs concentrated from culture supernatants by ultracentrifugation (FIG. 3B). Anti-Vpx antibody detected Vpx2, Vpx2SN and Vpx2SN* in cell lysates (top panel) and in VLPs derived by coexpression with pTM-gag2 (middle panel). Anti-Gag antibody detected p55 Gag in VLP pellets (bottom panel). Comparison of the relative protein signal intensities suggested that the Vpr1- and Vpx2- SN and SN* fusion proteins were packaged into VLPs in amounts similar to wild-type Vpr1 and Vpx2 proteins. Sucrose gradient analysis of VLPs containing Vpr1SN and Vpx2SN demonstrated co-sedimentation of these fusion proteins with VLPs (data not shown).

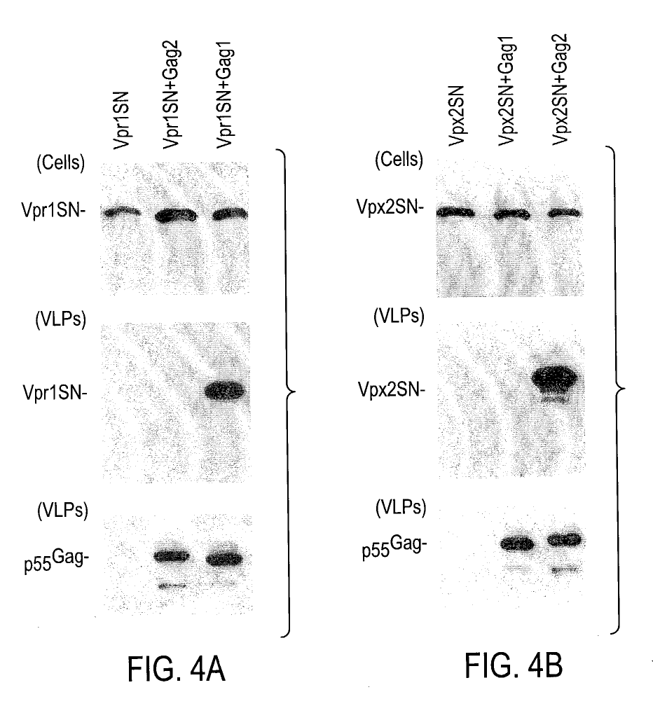

The Gag C terminal region is required for incorporation of Vpr1 and Vpx2 into virions. However, packaging was found to be virus type-specific, that is, when expressed in trans, Vpx2 was only efficiently incorporated into HIV-2 virions and HIV-2 VLPs. Similarly, HIV-1 Vpr required interaction with the HIV-1 Gag precursor for incorporation into HIV-1 VLPs. To show that the association of Vpr1SN and Vpx2SN with VLPs was not mediated by the SN moiety, but was due to the Vpr and Vpx specific packaging signals, pTM-vpr1SN and pTM-vpr2SN were cotransfected individually with either pTM-gag1 or pTM-gag2. For control, pTM-vpr1 and pTM-vpx2 were also transfected alone. Twenty-four hours later, lysates of cells and pelleted VLPs were examined by immunoblotting (FIG. 4). While Vpr1SN was expressed in all cells (FIG. 4A, top panel), it was only associated with VLPs derived from cells transfected with pTM-gag1. Similarly, Vpx2SN was detected in all pTM-vpx2 transfected cells (FIG. 4B, top panel), but was only associated with VLPs derived by cotransfection with pTM-gag2 (FIG. 4B, middle panel). HIV-1 and HIV-2 Gag monoclonal antibodies confirmed the presence of Gag precursor protein in each VLP pellet (FIG. 4B, bottom panels). Thus, incorporation of VpriSN and Vpx2SN into VLPs requires interaction of the cognate Gag precursor protein, just like native Vpr1 and Vpx2.

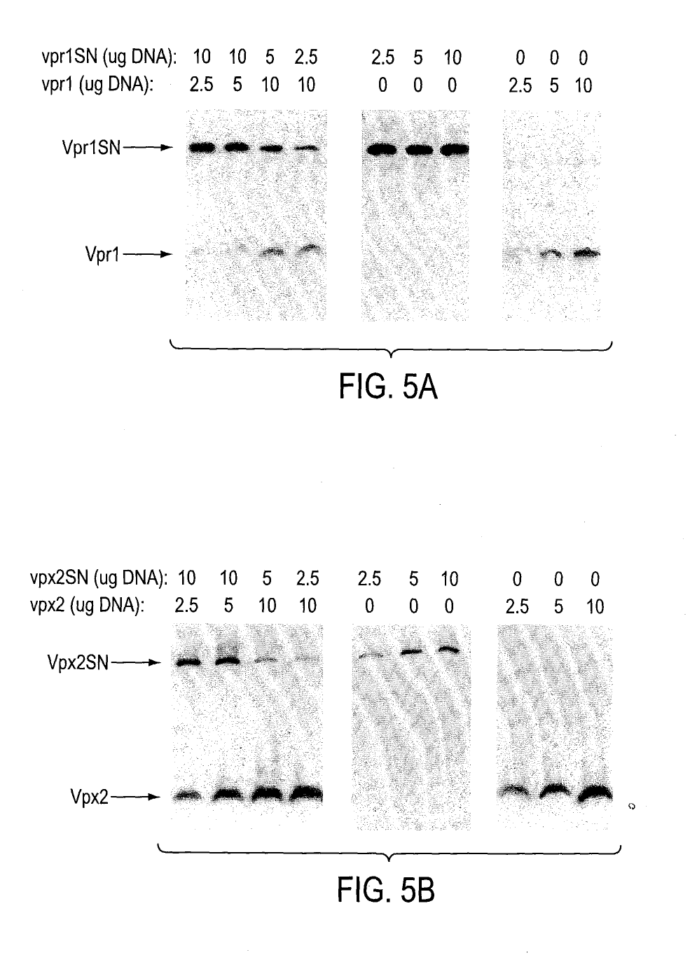

While Vpr1SN and Vpx2SN fusion proteins clearly associated with VLPs (FIG. 3), the question remained whether they would continue to do so in the presence of the native accessory proteins. The efficiency of Vpr1SN and Vpx2SN packaging was compared by competition analysis (FIG. 5). pTM-vpr1SN and pTM-vpx2SN were cotransfected with pTM-gag1/pTM-vpr1 and pTM-gag2/pTM-vpx2, respectively, using ratios that ranged from 1:4 to 4:1 (FIG. 5A and FIG. 5B, left panels). For comparison, pTM-vpr1 SN and pTM-vpr1 were transfected individually with pTM-gag1 (FIG. 5A, middle and right panels respectively) and pTM-vpx2SN and pTM-vpx2 were transfected with pTM-gag2 (FIG. 5B, middle and right panels respectively). VLPs were pelleted through sucrose cushions, lysed, separated by PAGE, blotted onto nitrocellulose and probed with anti-SN antibody. The results revealed the presence of both Vpr1 and Vpr1SN in VLPs when cotransfected into the same cells (FIG. 5A, left panel). Similarly, coexpressed Vpx2 and Vpx2SN were also copackaged (FIG. 5B, left panel). Comparison of the relative amounts of VLP-associated Vpr1SN and Vpx2SN when expressed in the presence and absence of the native protein, indicated that there were no significant packaging differences. Thus, Vpr1/Vpx2 fusion proteins can efficiently compete with wild-type proteins for virion incorporation.

### EXAMPLE 10

### Vpr1SN and Vpx2SN Fusion Proteins Possess Nuclease Activity

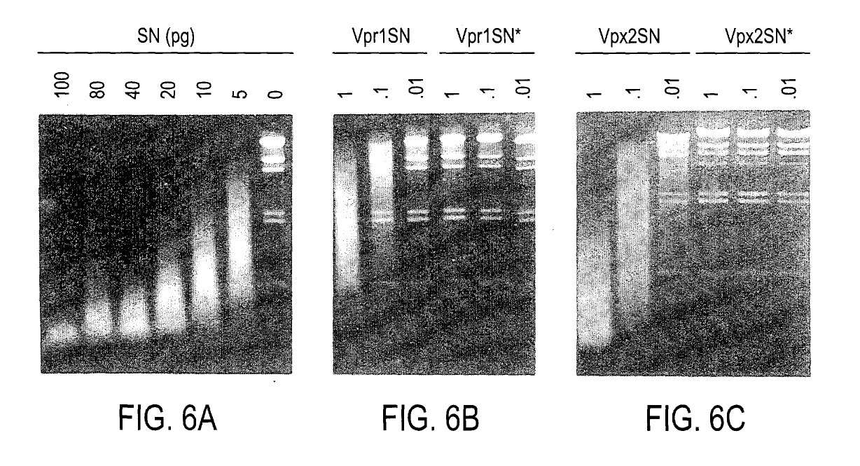

To demonstrate that virion associated SN fusion proteins were enzymatically active, VLPs concentrated by ultracentrifugation from culture supernatants of HeLa cells transfected with pTM-gag1/pTM-vpr1SN and pTM-gag2/pTM-vpx2SN were analyzed for nuclease activity using an in vitro DNA digestion assay. Prior to this analysis, immunoblotting confirmed the association of Vpr1SN and Vpx2SN with VLPs (data not shown). FIG. 6 shows lambda phage DNA fragments in 0.8% agarose gels after incubation with dilutions of VLPs lysates that contained Vpr1- or Vpx2-SN fusion proteins. VLPs containing Vpr1SN* and Vpx2SN* were included as negative controls and dilutions of purified SN served as reference standards (FIG. 6A). Both virion associated Vpr1 SN (FIG. 6B) and Vpx2SN (FIG. 6C) fusion proteins exhibited nuclease activity as demonstrated by degradation of lambda phage DNA. Cell-associated Vpr1SN and Vpx2SN fusion proteins also possessed nuclease activity when analyzed in this system (data not shown). To control for SN specificity, this analysis was also conducted in buffers devoid of Ca⁺⁺ and under these conditions no SN activity was detected (data not shown). Thus, SN remains enzymatically active when expressed as a fusion protein and packaged into VLPs.

### EXAMPLE 11

### Incorporation of Vpx2SN Fusion Protein into HIV-2 Virions

Vpx is incorporated into HIV-2 virions when expressed in trans. To show that Vpx2 fusion proteins were similarly capable of packaging into wild-type HIV-2 virions, an expression plasmid (pLR2P) was constructed placing the vpx2SN and vpx2SN* coding regions under control of HIV-2 LTR and RRE elements. The HIV-2 RRE was positioned downstream of the fusion genes to ensure mRNA stability and efficient translation (FIG. 7A). To show that the fusion proteins could package when expressed in trans, HIV-2_{ST} proviral DNA (PSXBI) was transfected alone and in combination with pLR2P-vpx2SN and pLR2P-vpx2SN*. Forty-eight hours later, extracellular virus as pelleted from culture supernatants by ultracentrifugation through cushions of 20% sucrose and examined by immunoblot analysis (FIG. 7B). Duplicate blots were probed using anti-Vpx (left), anti-SN (middle) and anti-Gag (right) antibodies. Anti-Vpx antibody detected the 15 kDa Vpx2 protein in all viral pellets. In virions derived by cotransfection of HIV-2_{ST} with pLR2P-vpx2SN and pLR2P-vpx2SN*, additional proteins of approximately 35 and 32 kDa were clearly visible. The same two proteins were also apparent on a duplicate blot probed with anti-SN antibodies, indicating that they represented Vpx2SN fusion proteins (FIG. 7B, middle panel). The predicted molecular weight of full-length Vpx2SN fusion protein is 33 kDa. As native Vpx and SN run slightly slower than predicted, it is likely that the 35 kDa species represents the full-length Vpx2SN fusion protein. Anti-SN antibodies detected additional proteins of approximately 21 and 17 kDa (these proteins were more apparent after longer exposure). Since only the 35 kDa protein was detected in Gag derived VLPs, which lack Pol proteins (FIG. 2), the SmaIler proteins represented cleavage products of Vpx2SN and Vpx2SN* generated by the viral protease. Anti-Gag antibodies confirmed the analysis of approximately equivalent amounts of virions from each transfection.

To show packaging of Vpx2SN into HIV-2 virions, sucrose gradient analysis was performed. Extracellular virus collected from culture supernatants of HLtat cells forty-eight hours after cotransfection with pLR2P-vpx2SN and HIV-2_{ST} was pelleted through cushions of 20% sucrose. Pellets were resuspended in PBS and then centrifuged for 18 hours over linear gradients of 20-60% sucrose. Fractions were collected and analyzed by immunoblotting (FIG. 7C). Duplicate blots were probed separately with anti-SN (top) and anti-Gag (bottom) antibodies. Peak concentrations of both Vpx2SN and Gag were detected in fractions 8-11, demonstrating direct association and packaging of Vpx2SN into HIV-2 virions. These same sucrose fractions (8-11) were found to have densities between 1.16 and 1.17 g/ml, as determined by refractometric analysis (data not shown). Again, both the 35 kDa and 32 kDa forms of Vpx2SN were detected, providing further evidence for protease cleavage following packaging into virus particles.

Since HIV-2_{ST} is defective in vpr, this may have affected the packaging of the Vpx2SN fusion protein. A second strain of HIV-2, termed HIV-2_{7312A}, was analyzed which was cloned from short-term PBMC culture and contains open reading frames for all genes, including intact vpr and vpx genes (unpublished). A plasmid clone of HIV-2_{7312A} proviral DNA (pJK) was transfected alone and in combination with pLR2P-vpx2SN into HLtat cells. For comparison, HIV-2_{ST} was also co-transfected with pLR2P-vpx2SN. Progeny virus was concentrated by ultracentrifugation through sucrose cushions and examined by immunoblot analysis (FIG. 7D). Duplicate blots were probed with anti-Vpx (left) and antiGag (right) antibodies. The results revealed comparable levels of Vpx2SN incorporation into vpr competent virus (HIV-2_{7312A}) compared with vpr-defective virus (HIV-2_{ST}). Moreover, the 35 kDa and 32 kDa proteins were again detected in HIV-2_{7312A} virions. Thus, efficient incorporation of the Vpx2SN protein into replication-competent wild-type HIV-2 was demonstrated, even in the presence of native Vpr and Vpx proteins.

### EXAMPLE 12

### Incorporation of Vpr1SN into HIV-1 Virions

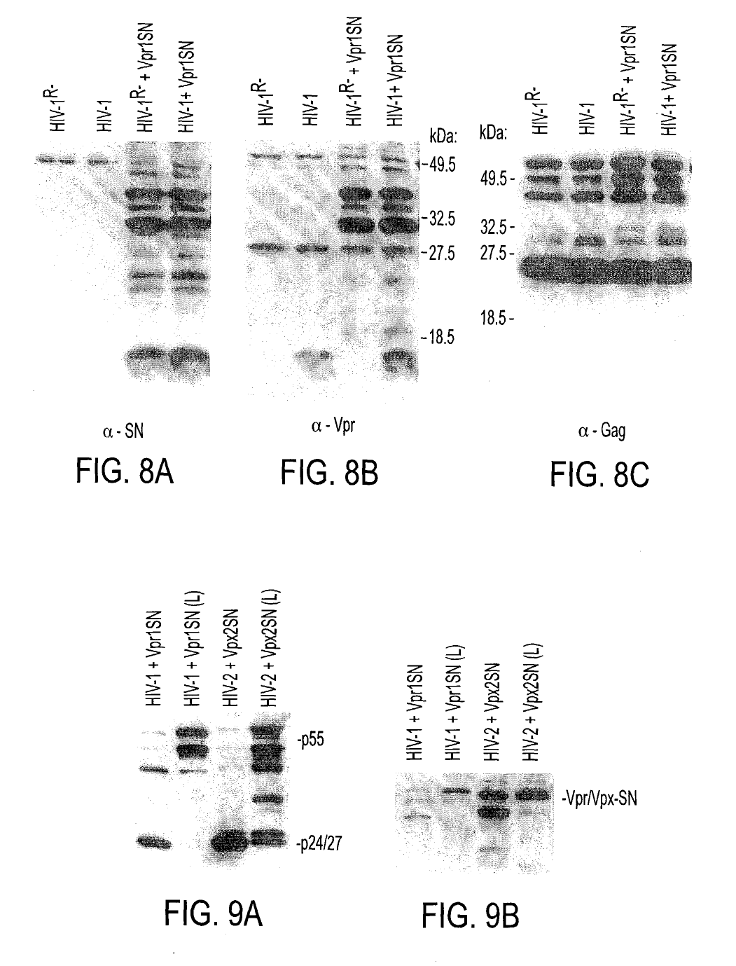

Using the same LTR/RRE-based expression plasmid, it was also shown that Vpr1SN could package into HIV-1 virions by co-expression with HIV-1 provirus (as discussed above, the HIV-2 LTR can be transactivated by HIV-1 Tat and the HIV-2 RRE is sensitive to the HIV-1 Rev protein). Virions released into the culture medium 48 hours after transfection of HLtat cells with pNLA4-3 (HIV-1) and pNL4-3-R (HIV-1-R) alone and in combination with pLR2P-vprISN were concentrated by ultracentrifugation and examined by immunoblot analysis (FIG. 8). As observed in cotransfection experiments with HIV-2, anti-SN antibodies identified two major Vpr1SN fusion proteins of approximately 34 to 31 kDa. These proteins were not detected in virions produced by transfection of pNL4-3 and pNL4-e-R⁻ alone. From expression in the rVT7 system, the full-length Vpr1 SN fusion protein was expected to migrate at 34 kDa. Therefore, the 31 kDa protein likely represents a cleavage product. Anti-SN antibodies also detected a protein migrating at 17 kDa. Anti-Vpr antibody detected the 34 and 31 kDa proteins in virions derived from cotransfected cells. It is noteworthy that both the anti-Vpr and anti-SN antibodies detected the 31 kDa protein most strongly, and that anti-Vpr antibody did not detect the 17 kDa protein recognized by anti-SN antibody. These results also show that even in virions in which native Vpr protein was packaged, Vpr1SN was also incorporated in abundance. Gag monoclonal antibody detected similar amounts of Gag protein in all viral pellets and demonstrated processing of the p55^{Gag} precursor (FIG. 8C).

To demonstrate more directly that cleavage of the Vpr1- and Vpx2-SN fusion proteins was mediated by the HIV protease, virus was concentrated from pNL4-3-R⁻/pLR2P-vpr1SN and pSXB1/pLR2P-vpx2SN transfected cells that were culture in the presence of 1 μM of the HIV protease inhibitor L-689,502 (provided by Dr. E. Emini, Merck & Co. Inc.). As expected, immunoblot analysis of virions demonstrated substantially less processing of p55^{Gag} (FIG. 9A). Similarly, virions produced in the presence of L-689,502 also contained greater amounts of the uncleaved species of Vpr1SN and Vpx2SN fusion proteins (FIG. 9B). Taken together, these results demonstrate that Vpr1- and Vpx2- SN fusion proteins are subject to protease cleavage during or subsequent to virus. assembly.

### EXAMPLE 13

### Vpr1-CAT and Vpr2-CAT Fusion Protein Incorporation into HIV Virions

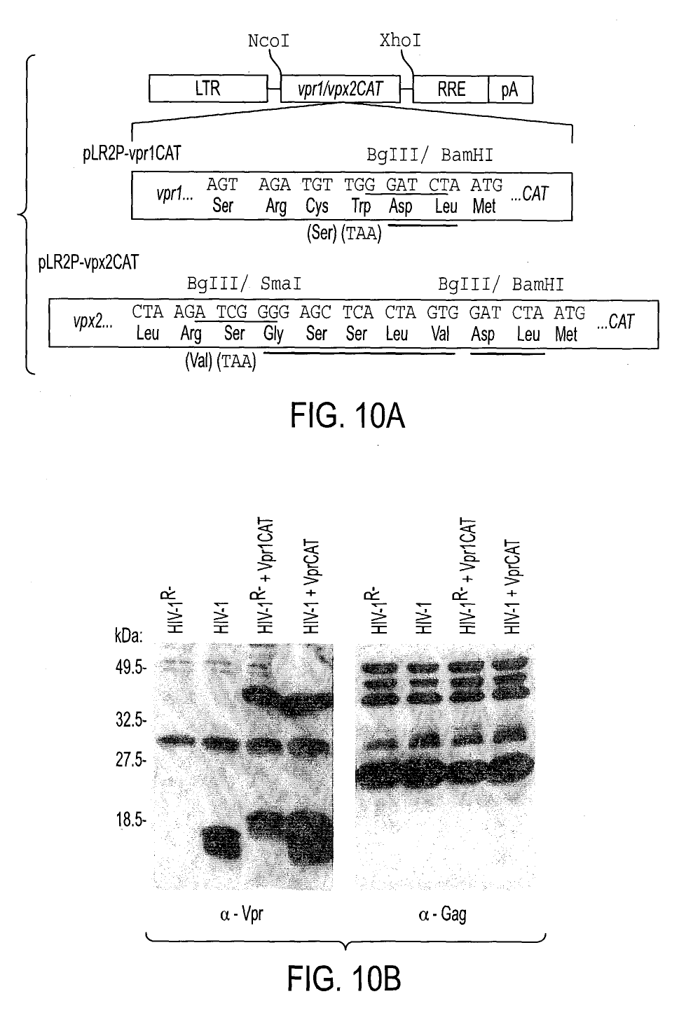

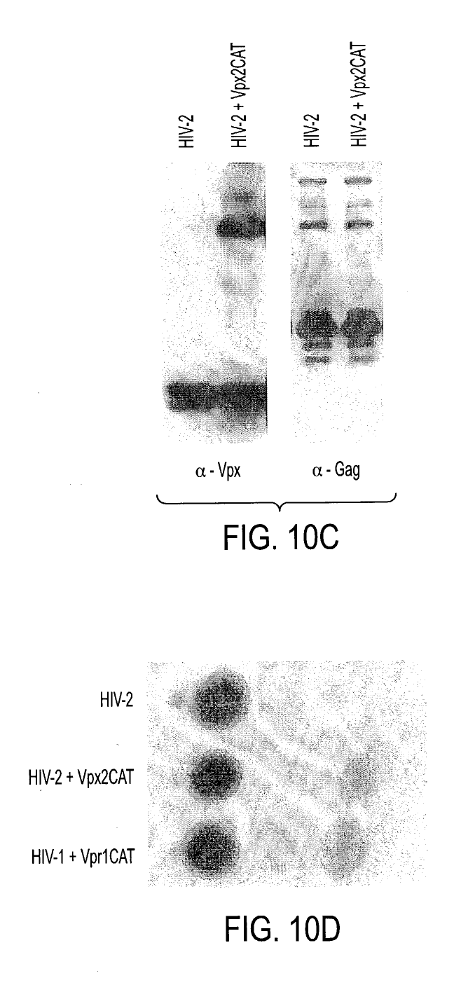

To show that Vpx2 and Vpr1 could target additional proteins to the HIV particle, the entire 740 bp CAT gene was substituted for SN in the pLR2P-vpx2SN and pLR2P-vpr1SN vectors, generating pLR2P-vpr1CAT and pLR2P-vpx2CAT (FIG. 10A). pNL4-3/pLR2P-vpr1CAT, pnl4-3-R⁻/pLR2P-vpr1CAT and pSXB1/pLR2P-vpx2CAT were co-transfected into HLtat cells. As controls, pNL4-3, pNL4-3-R⁻ and pSXBI were transfected alone. Progeny virions, concentrated from culture supernatants, were analyzed by immunoblotting (FIGS. 10B and 10C). Using anti-Vpr antibodies, 40 kDa fusion proteins were detected in viral pellets derived by co-transfection of pRL2P-vpr1CAT with both pNL4-3 and pNL4-3-R⁻ (FIG. 10B). This size is consistent with the predicted molecular weight of the full-length Vpr1CAT fusion protein. In addition, anti-Vpr antibodies also detected a 17 kDa protein which did not correspond to the molecular weight of native Vpr1 protein (14.5 kDa in virions derived from cells transfected with pNL4-3). The same protein was recognized weakly with antiCAT antibodies, suggesting a fusion protein cleavage product containing most Vpr sequence. Very similar results were obtained with virions derived from HLtat cells co-transfected with HIV-2_{ST} and pRL2P-vpx2CAT, in which anti-Vpx antibody detected 41 and 15 kDa proteins (FIG. 10C). These results demonstrate that Vpr1CAT and Vpx2CAT fusion proteins are packaged into virions. However, like in the case of SN fusion proteins, CAT fusion proteins were also cleaved by the HIV protease (the Vpx2CAT cleavage product is not visible because of co-migration with the native Vpx protein. CAT cleavage appeared less extensive, based on the intensity of the full-length CAT fusion protein on immunoblots.

Lysates of HIV-1 and HIV-2 viral particles were diluted 1:50 in 20 mM Tris-base and analyzed for CAT activity by the method of Allon, et al., Nature 282:864-869 (1979). FIG. 10D indicates that virions which contained Vpr1CAT and Vpx2CAT proteins possessed CAT activity. These results show the packaging of active Vpr1- and Vpx2-CAT fusion proteins.

### EXAMPLE 14

### Virion Incorporated SN and CAT Fusion Proteins are Enzymatically Active

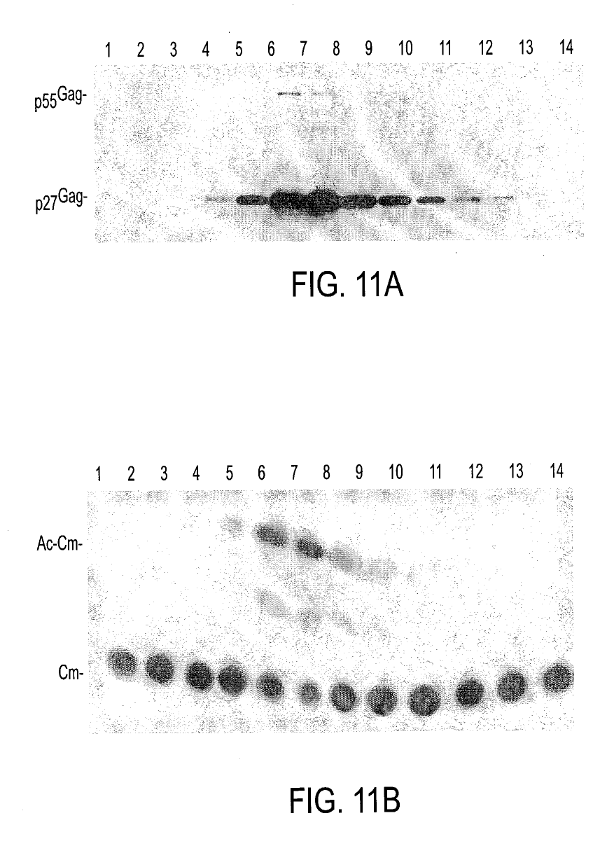

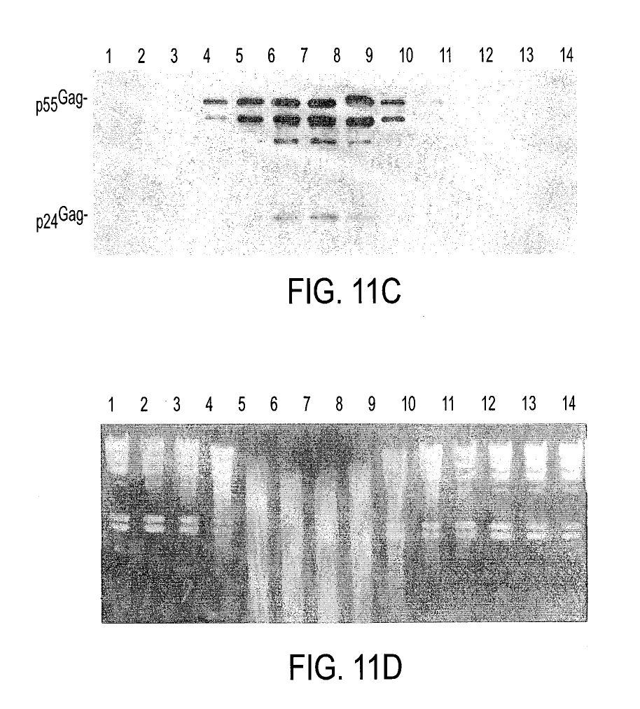

The ability of Vpr1 and Vpx 2 to deliver functionally active proteins to the virus particle was further confirmed by sucrose gradient analysis. Virions derived from HLtat cells co-transfected with HIV-2_{ST} and pLR2P-vpx2 were sedimented in linear gradients of 20-60% sucrose as described above. Fractions were collected and analyzed for viral Gag protein (FIG. 11) and corresponding CAT activity (FIG. 11B). Peak amounts of Gag protein were detected in fractions 6 and 7 (density 1.16 and 1.17, respectively). Similarly, peak amounts of acetylated chloramphenicol (Ac-cm) were also detected in fractions 6 and 7.

Whether virion associated SN fusion protein retained nuclease activity was also shown. HIV-1_{SG3} virions containing Vpr1SN were analyzed after sedimentation in linear gradients of sucrose (FIG. 11). Since the present invention demonstrated that protease cleavage of SN fusion proteins (FIGS. 7, 8 and 9) markedly reduced Vpr1SN nuclease activity (data not shown), these experiments were performed by culturing pSG3/pLR2P-vpr1SN co-transfected cells in the presence of L-689,502 as described above. Immunoblot analysis of sedimented virions revealed peak concentrations of Gag in fractions 6 and 7 and substantially reduced p55 processing (FIG. 11C). Peak SN activity was associated with the fractions that contained the highest concentrations of virus (FIG. 11D). These results thus document that virion incorporation per se does not abrogate the enzymatic activity of Vpr/Vpx fusion proteins, although cleavage by the viral protease may inactivate the fusion partners.

### EXAMPLE 15

### Construction and Design of a Gag-Pro (RT-IN Minus) Packaging Plasmid

Several different strategies have been used to express Gag-Pro. Placing Gag and Pro in the same reading frame leads to over expression of Pro and marked cell toxicity. It is known that deletions within the RT and IN coding regions, including smaller deletion mutations, may cause marked defects in the expression levels of the Gag-Pro and Gag-Pol proteins, respectively (1, 2, 4, 7, 23). Importantly, the viral particles produced under these circumstances are defective in proteolytic processing and are not infectious, even if RT and IN are provided in trans (28). The reduced levels of expression and virion associated Gag-Pol protein is apparently due to an effect on the frequency of Gag-Pol frame-shifting. Gag-Pol frame-shifting is not markedly affected when the translation of RT and IN is abrogated, which is distinct from deletions of viral DNA fragment. Virions which assembly Gag-Pro, when RT and IN protein synthesis is abrogated by a translational stop codon, mature and are infectious when RT and IN are provided in trans (28). Therefore, a Gag-Pro packaging plasmid of the present invention is preferably constructed by abrogating translation of sequence downstream of Pro (RT-IN). Other mutations in Gag and Pol would also function as part(s) of the trans-lentiviral packaging system if they did not cause major defects in particle assembly and infectivity. In addition to introducing a translational stop codon (TAA) at the first amino acid residue of RT, at least one addition “fatal” mutation is positioned within RT and IN (FIG. 12B). This mutation further decreases the likelihood of reestablishing a complete Gag-Pol coding region by genetic recombination between packaging (gag-pro) and enzymatic (vpr-RT-IN) plasmids. It is appreciated that the stop codon can be inserted within the gene sequence in a position other than at the first codon for the first amino acid residue of a protein and still be an effective measure to prevent infectivity. A stop codon generally inserted with the front half of the amino acid encoding nucleic acid residues is effective, although the stop codon is preferentially at the beginning of the translational sequence. A fatal mutation as used herein refers to a mutation within the gene sequence that render the coded polypeptide sequence functionally ineffectual in performing the biological role of the wild protein.

The Gag-Pro expression plasmid (pCR-gag-pro) includes the CMV promoter and the HIV-2 Rev responsive element (RRE) (FIG. 12C). The RRE allows for the efficient expression of HIV proteins (including Gag, PR, RT, IN) that contain MRNA inhibitory sequences. RT and IN are provided by transexpression with the pLR2P-vpr-RT-IN expression plasmid (FIG. 12C). This vector expresses the Vpr-RT-IN fusion protein which is incorporated into HIV-1 virions/vector in trans, and is proteolytically processed by the viral protease to generate functional forms of RT (p51 and p66) and IN (28). This earlier work shows that functional RT and IN can be provided separately (Vpr-RT and Vpr-IN) (12, 28). Preferably, the Vpr component of the fusion protein contains a His71Arg substitution which knocks out the Vpr cell cycle arrest function.

### EXAMPLE 16

### Production of the Trans-Lentiviral Vector

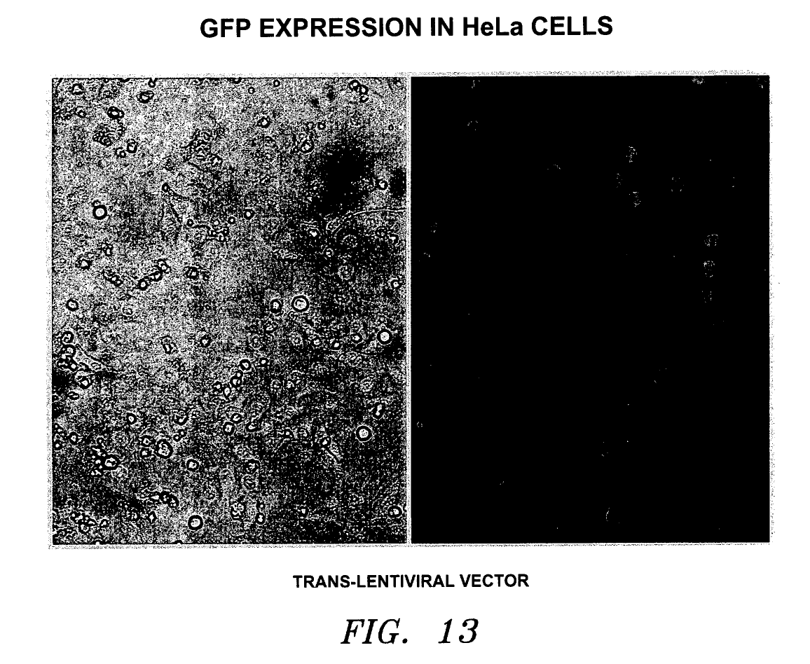

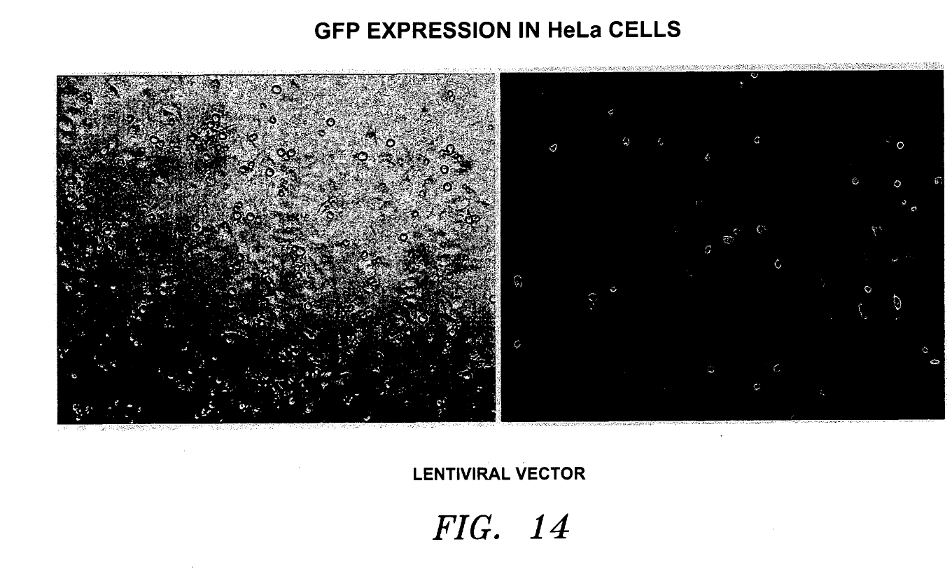

4 ug each of pCR-gag-pro, pLR2P-vpr-RT-IN (enzymatic plasmid), pHR-CMV-β-gal (marker gene transduction plasmid) and pCMV-VSV-G (env plasmid) were transfected into 293T cell line. 293T cells were used since they produce high titered stocks of HIV particles/vector and are exquisitely sensitive to transfection, including multiple plasmid transfections. As a control, in side-by-side experiments, the pΔ8.2 packaging plasmid was also transfected with PHR-CMV-β-gal and pCMV-VSV-G (FIG. 12B). The pΔ8.2 plasmid is a lentivirus packaging vector obtained from Dr. D. Trono. The pΔ8.2 produces high titered vector stocks upon transfection with pHR-CMV-β-gal and pCMV-VSV-G (16, 34), (approximately 1-5×10⁵ infectious particles/ml supernatant, with a p24 antigen concentration of 150-800 ng/ml). Approximately 72 hours after transfection, the culture supernatants were harvested, clarified by low-speed centrifugation, filtered through a 0.45 micron filter, and analyzed for p24 antigen concentration by ELISA. To examine the titer of the trans-lentiviral vector, supernatant stocks of 25, 5, 1, and 0.2 ul were used to infect cultures of HeLa cells and IB3 cells. Two days later, the cells are stained with X-gal, and positive (blue) cells are counted using a light microscope. Table 1 shows that the titer of trans-lentiviral vector. These results show that the trans-lentiviral vector can achieve titers as high as 2×10⁵/ml, although its titer is consistently lower than that of lentiviral vector (2-5 folds less). For direct examination of transduction in living cells the transduction plasmid was also constructed to contain the GFP gene/marker (FIGS. 12B and 12C). Stocks of trans-lentiviral and lentiviral vector were produced as described above and used to infect HeLa cells. Two days later the cells were examined by fluorescence microscopy. FIGS. 13 and 14 show positive gene transduction with the trans-lenti and lentiviral vectors respectively.

### EXAMPLE 17

### Concentration of Trans-Lentiviral Vector by Ultracentrifugation

To examine whether the trans-lentiviral vector was stable during the concentration by ultracentrifugation, the supernatant-trans-lentiviral vector was concentrated by ultracentrifugation (SW28, 23,000 rpm, 90 min., 4 deg. C.). As a control supernatant-lentiviral vector was concentrated in parallel. The titers for both were determined both before and after concentration. Table 2 shows our results and indicates that the trans-lentiviral vector is stable during concentration by ultracentrifugation.

### EXAMPLE 18

### Trans-Lentiviral Vector for CFTR Gene Transduction

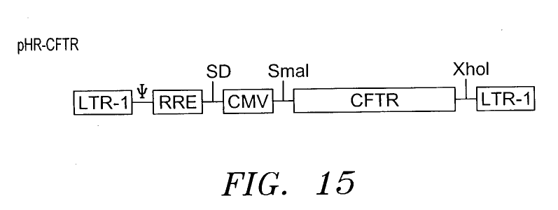

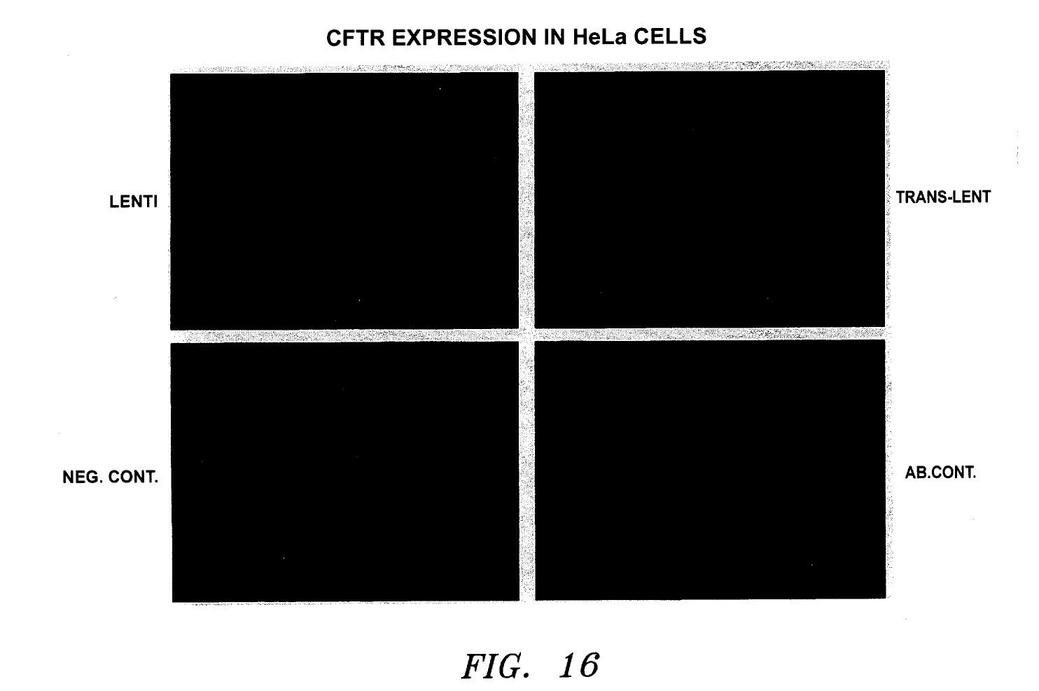

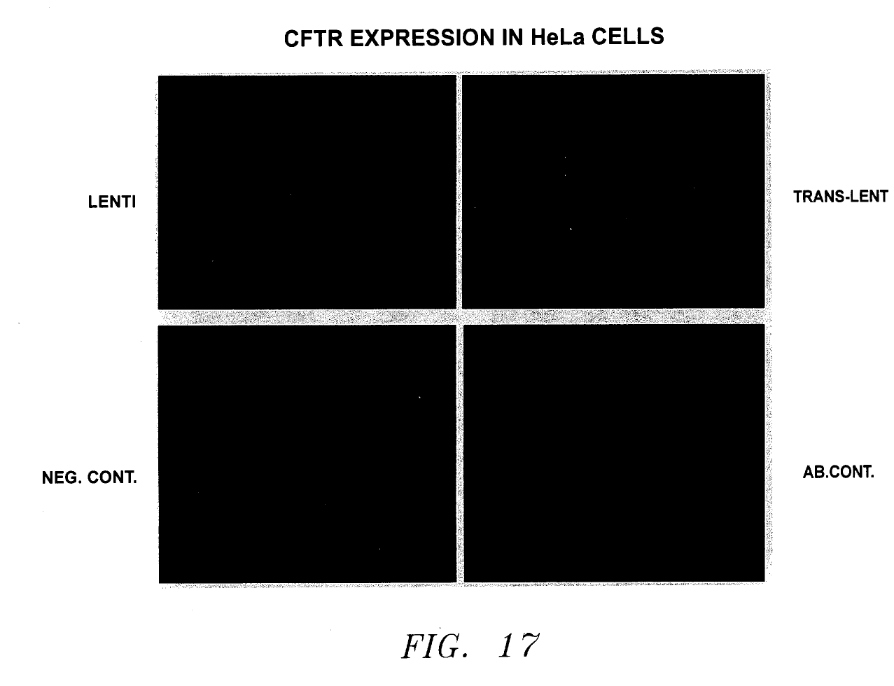

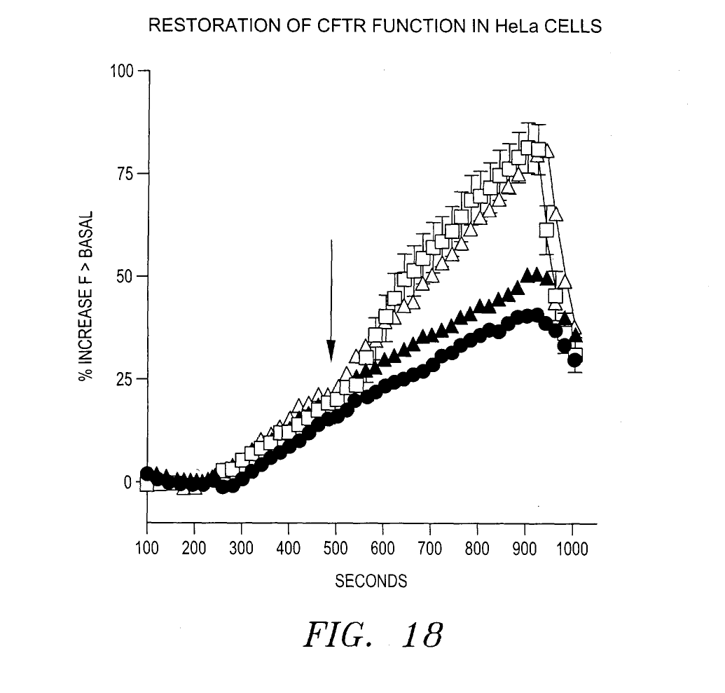

Lentiviral-based vectors are attractive for use in the lung due to their ability to transduce non-divided cells. This unique characteristic may represent an important advantage of lentiviral vectors for gene therapy of CF. A translentiviral vector was used to deliver the CFTR gene into HeLa cells. The CFTR gene was cloned into the pHR transduction plasmid, using SmaI and XhoI sites (FIG. 15). Trans-lentiviral and lentiviral (as control) vectors were generated by transfection as described above, and used to transduce HeLa cells grown on cover slips. Two days later the cells were examined by immunofluorescence microscopy, using both polyclonal (FIG. 16) and monoclonal antibodies (FIG. 17). The results show CFTR expression and localization of CFTR on the cell surface. Furthermore, the transduced HeLa cells examined by SPQ (halidesensitive fluorophore) showed restored CFTR function (FIG. 18).

The present invention demonstrated the capability of HIV-1 Vpr and HIV-2 Vpx to direct the packaging of foreign proteins into HIV virions when expressed as heterologous fusion molecules. The trans complementation experiments with HIV proviral DNA revealed that Vpr1 and Vpx2 fusion proteins were also incorporated into replication-competent viruses. Moreover, packaging of the fusion proteins in the presence of wild-type Vpx and/or Vpr proteins (FIGS. 16 and 17) indicated that the viral signals mediating their packaging were not obstructed by the foreign components of the fusion molecules. Likewise, virion-associated SN and CAT fusion proteins remained enzymatically active.

Based on the immunoblot analysis of VLPs and virions, the present invention illustrates that both virion associated CAT and SN/SN* are susceptible to cleave by the viral protease. There appears to be at least one cleavage site in CAT and two cleavage sites in the SN/SN* proteins. Based on calculated molecular weights of the major SN/SN* cleavage products, it appears that SN and SN* are cleaved one near their C termini and once near the fusion protein junctions. Since the fusion protein junctions of Vpr1SN and Vpx2SN are not identical it is also possible that these regions differ with respect to their susceptibility to the viral protease. Although Vpx2SN/SN* were processed to a lesser extent than Vpr1SN (FIGS. 7 and 8), the major cleavage sites appear to be conserved. There is no doubt that both the HIV-1 and HIV-2 proteases recognize processing sites in the fusion partners and that there is sufficient physical contact to enable cleavage. This is evidenced both by the reduction of cleavage product intensities on immunoblots as well as by an increased enzymatic activity in the presence of an HIV protease inhibitor.

The demonstration that Vpr1 and Vpx2 fusion proteins are capable of associating with both VLPs and virions facilitates studies on these accessory proteins and on HIV assembly in general. The approach of generating deletion mutants to study protein structure/function relationships is often of limited value since this can reduce protein stability or change the three-dimensional structure of the protein. In the case of Vpr, a single amino acid substitution at residue 76 has been shown to destabilize its expression in infected cells. Studies have indicated that deletion mutations in vpr and vpx result in premature degradation of the proteins following expression. Fusion of Vpr and Vpx mutant proteins with, e.g., SN or CAT as demonstrated by the present invention, increase stability.

The successful packaging of Vpr1/Vpx2SN fusion proteins into virions indicates their use for accessory protein targeted viral inactivation. The present invention demonstrates that Vpr and Vpx may serve as vehicles for specific targeting of virus inhibitory molecules, including SN. In contrast to HIV Gag, Vpr and Vpx are small proteins that can be manipulated relatively easily without altering virus replication and thus may represent vehicles with considerable versatility for application to such an antiviral strategy.

### EXAMPLE 19

### Incorporation of RT in Trans into a Lentivirus Independent of HIV Accessory Proteins

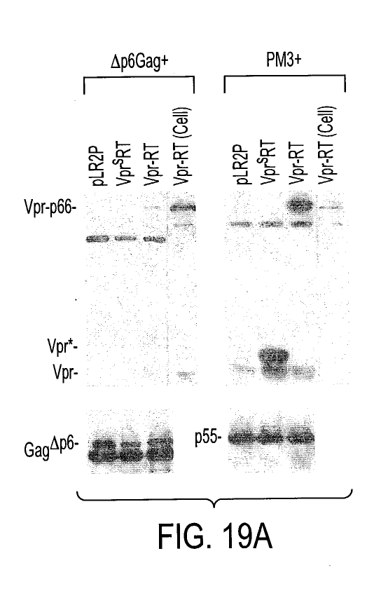

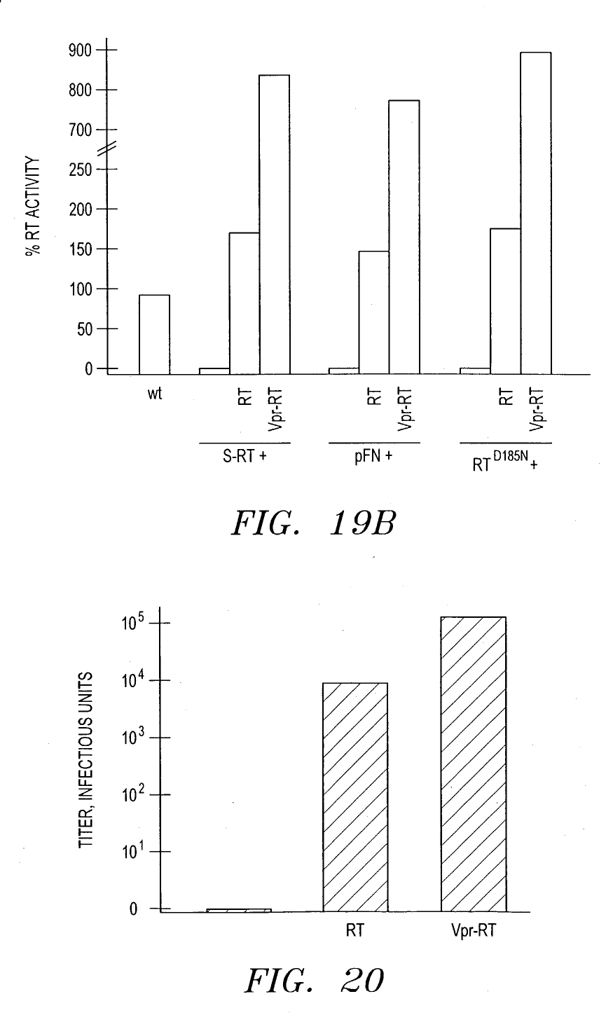

The HIV accessory proteins, Vpr and Vpx, are incorporated into virions through specific interactions with the p6 portion of the Pr55^{Gag} precursor protein (Kappes et al., 1993; Kondo et al., 1995; Lu et al., 1995; Paxton et al., 1993; Wu et al., 1994). Similarly, it has been demonstrated that Vpr and Vpx fusion proteins (Vpr- and Vpx- SN and CAT) are incorporated into virions through interactions with p6^{Gag}, similar to that of the wild-type Vpr and Vpx proteins (Wu et al., 1995). To analyze the contribution of Vpr for incorporation of the Vpr-RT fusion protein into virions, an HIV-1 proviral clone mutated in p6^{Gag} and PR (designated pNL43-Δ p6^{Gag}, provided by Dr. Mingjun Huang) was cotransfected with pLR2P-vprRT into 293T cells. This mutant contains a TAA translational stop codon at the first amino acid residue position of p6^{Gag}. This abrogated the Gag sequences that are required for Vpr virion incorporation. The pNL43-Δ p6^{Gag} clone also contains a mutation (D25N) in the active site of PR, which enhances the release of the p6 Gag mutant virus from the cell surface membrane (Göttlinger et al., 1991; Huang et al., 1995). As a control, the HIV-1 PR mutant PM3 (Kohl et al., 1988), derived from the same pNL4-3 parental clone, was also included for analysis. Progeny virions, purified from pNL43-66 p6^{Gag} transfected cell cultures, contained detectable amounts of RT protein (labeled as Vpr-p66), albeit in lesser amounts compared with virions derived from PM3 (FIG. 19). Analysis of cell lysates confirmed expression, and compared with PM3, the accumulation of Vpr-RT in pLR2P-vprRT/pNL43-Δ p6^{Gag} cotransfected cells. Vpr^{S}-RT was included as an additional control and was shown to incorporate Vpr efficiently into PM3 virions but not into those derived by coexpression with pNL43-Δ p6^{Gag}. Wild-type Vpr protein was also absent from Δ p6^{Gag} virions. Approximately equal amounts of Gag protein was detected in the different virus pellets, confirming that similar amounts of the different virions were compared in the quantitation. These results show that RT protein can be incorporated into virions independently of Vpr-p6 mediated interaction. These data also indicate that expression of RT (and IN by inference) in trans, independently of Gag-Pol, is sufficient for its incorporation and function.

### EXAMPLE 20

### Expression of RT in Trans in a Lentivirus Vector Independent of HIV Accessory

It has been demonstrated that functional RT can be incorporated into HIV-1 virions by its expression in trans, even without fusion to Vpr (Example 19). To determine if RT expressed in trans can package into lentiviral vector and support the transduction of a marker gene RT was ligated into the pLR2P expression plasmid under control of the HIV LTR and RRE, generating the pLR2P-RT expression plasmid. The pLR2P-RT, pHR-CMV-VSV-G, pHR-CMV-β-gal, and pΔ8.2-RTD^{D185N} was transfected together into 293T cells. The pΔ8.2-RT^{D185N} plasmid contains a point mutation in RT at amino acid residue position 185 (D185N), which abolishes polymerase activity and destroys its ability to support gene transduction. As a control Vpr-RT (pLR2P-vpr-RT) was substituted for pLR2P-RT in a parallel experiment. As another control neither RT or Vpr-RT 46 were provided. Virions generated by transfection were used to infect HeLa cells. Two days later, transduction positive cells were counted. FIG. 20 shows that both Vpr-RT and RT support vector transduction when provided in trans. The vector titer was reduced by about 10-fold when RT was provided without fusion with Vpr. These results demonstrate that enzymatic function (RT and IN) can be provided in trans, independently of Gag-Pol.

The present invention demonstrated that Vpr and Vpx can serve as vehicles to deliver functionally active enzymes to the HIV virion, including those that may exert an antiviral activity such as SN. The present invention has demonstrated that the concept of accessory protein targeted virus inactivation is feasible.

Any patents or publications mentioned in this specification are indicative of the levels of those skilled in the art to which the invention pertains. These patents and publications are herein incorporated by reference to the same extent as if each individual publication was specifically and individually indicated to be incorporated by reference.

### REFERENCES

1. Ansari-Lari, M. A. et al. 1995. Virology. 211:332-335.

2. Ansari-Lari, M. A. et al. 1996. Journal of Virology. 70:3870-3875.

3. Baba, T. W. et al. 1995. Science. 267:1820-1825.

4. Bukovsky, A. et al. 1996. Journal of Virology. 70:6820-6725.

5. Bukrinsky, M. I. et al.—1993. Nature (London). 365:666-669.

6. Endres, M. J. et al. 1997. Science. 278:1462-1464.

7. Engelman, A. et al. 1995. Journal of Virology. 69:2729-2736.

8. Heinzinger, N. K. 1994. Proc. Natl. Acad. Sci. USA. 91:7311-7315.

9. Kimpton, J. et al. 1992. Journal of Virology. 66:2232-2239.

10. Kondo, E. et al. 1995. J. Virol. 69:2759-2764.

11. Liu, H. et al. 1995. J. Virol. 69:7630-7638.

12. Liu, H. et al. 1997. J. Virol. 71:7704-771 G.

13. Lu, Y.-L. et al. 1995. Journal of Virology. 69:6873-6879.

14. Mebatsion, T. et al. 1997. Cell Press. 90:841-847.

15. Naldini, L. et al. 1996. Science. 272:263-267.

16. Naldini L. et al. 1996. Science. 272:263-267.

17. Pathak, V. K. et al. 1997. Academic Press. 1044:141-150.

18. Paxton, W. et al. 1993. J. Virol. 67:7229-7237.

19. Rogel, M. E. et al. 1995. Journal of Virology. 69:882-888.

20. Ruprecht, R. M. et al. 1996. Science. 271:1790-1792.

21. Ruprecht, R. M. et al. 1996. AIDS Research & Human Retroviruses. 12:459-460.

22. Schnell, M. J. et al. 1997. Cell Press. 90:849-857.

23. Shin, C.-G. et al. 1994. Journal of Virology. 68:1633-1642.

24. Stewart, S. A. et al. 1997. Journal of Virology. 71:5579-5592.

25. Verma, I. M. et al. 1997. Nature. 389:239-242.

26. von Schwedler, U. et al. 1994. Proc. Natl. Acad. Sci. USA. 91:66926696.

27. Wu, X. et al. 1994. J. Virol. 68:6161-6169.

28. Wu, X. et al. 1997. EMBO Journal. 16:5113-5122.

29. Wu, X. et al. 1996. Journal of Virology. 70:3378-3384.

30. Wu, X. et al. 1995. J. Virol. 69:3389-3398.

31. Yao, X.-J. et al. 1995. Journal of Virology. 69:7032-7044.

32. Zhang, J. et al. 1993. Journal of Virology. 67:1747-175 1.

33. Zhang, J. et al. 1993. Science. 259:234-238.

34. Zufferey, R. et al. 1997. Nature Biotechnology. 15:871-875.

One skilled in the art will readily appreciate that the present invention is well adapted to carry out the objects and obtain the ends and advantages mentioned, as well as those inherent therein. The present examples along with the methods, procedures, treatments, molecules, and specific compounds described herein are presently representative of preferred embodiments, are exemplary, and are not intended as limitations on the scope of the invention. Changes therein and other uses will occur to those skilled in the art which are encompassed within the spirit of the invention as defined by the scope of the claims.

TABLE 1
Generation of trans-lentiviral vector

Titer (inf. units/ml × 10⁻⁵)

| packaging plasmid | RT-TN plasmid | HeLa | IB3 |
|---|---|---|---|
| pCMVΔR9 | — | 2.5 (+/−5.1) | 1.2 (+/−2.7) |
| pCMVΔR9-S^{RT-IN} | — | 0 | 0 |
| pCMVΔR9-S^{RT-IN} | Vpr-RT-IN | 1.1 (+/−3.1) | 0.8 (+/−2.5) |

TABLE 2
Generation of trans-lentiviral vector

Titer (inf.
units/ml × 10⁻⁵)

| packaging plasmid | RT-IN plasmid | HeLa | IB3 |
|---|---|---|---|
| pCMVΔR9 | — | 54 | 31 |
| pCMVΔR9-S^{RT-IN} | Vpr-RT-IN | 28 | 19 |

That which is claimed:

1. A viral vector system comprising:
a) at least a first nucleic acid segment comprising a nucleotide sequence encoding at least a functional portion of a Gag polypeptide, and said first nucleic acid segment does not encode at least one of a functional Reverse Transcriptase polypeptide and a functional Integrase polypeptide; and,
b) at least a second nucleic acid segment comprising at least one nucleotide sequence encoding a polypeptide selected from the group consisting of:
i) a functional portion of a Reverse Transcriptase polypeptide; and,
ii) a functional portion of an Integrase polypeptide;
wherein said second nucleic acid segment does not encode a functional Gag polypeptide; and,
c) at least a third nucleic acid segment comprising a nucleic acid sequence encoding a functional portion of an envelope polypeptide, wherein said third nucleic acid segment does not encode a functional Gag-Pol precursor polypeptide;
wherein said viral vector system produces an infectious viral particle.

2. The viral vector system of claim 1, wherein
said first nucleic acid segment does not encode the functional Reverse Transcriptase and the functional Integrase polypeptide;
said second nucleic acid segment comprises a nucleotide sequence encoding the functional portions of the Reverse Transcriptase polypeptide fused in frame to the functional portions of the Integrase polypeptide.

3. The viral vector system of claim 1, wherein
said first nucleic acid segment does not encode the functional Reverse Transcriptase and the functional Integrase polypeptide;
said second nucleic acid segment comprises a nucleotide sequence encoding a functional portion of the Reverse Transcriptase polypeptide; and,
said viral vector system further comprises a fourth nucleic acid segment comprising a nucleotide sequence encoding a functional portion of the Integrase polypeptide, wherein said fourth nucleic acid segment does not encode the functional Gag polypeptide.

4. The viral vector system of claim 1, wherein said first nucleic acid segment further comprises a Protease polypeptide.

5. The viral vector system of claim 1 further comprising at least a fourth nucleic acid segment comprising a nucleic acid sequence of interest, wherein said fourth nucleic acid segment does not encode a functional Gag-Pol precursor polypeptide wherein said viral vector system produces an infectious viral particle capable of transducing a target cell.

6. The viral vector system of claim 3 further comprising at least a fifth nucleic acid segment comprising a nucleic acid sequence of interest, wherein said fifth nucleic acid segment does not encode a functional Gag-Pol precursor polypeptide wherein said viral vector system produces an infectious viral particle capable of transducing a target cell.

7. The viral vector system of claim 5, wherein said nucleotide sequence of interest encodes a polypeptide.

8. The viral vector system of claim 7, wherein said polypeptide is a viral inhibitory polypeptide.

9. The viral vector system of claim 5, wherein said nucleotide sequence of interest is operably linked to a promoter active in a target cell.

10. The viral vector system of claim 1, wherein said functional portions of said Gag polypeptide, said Reverse Transcriptase polypeptide, and said Integrase polypeptide are from a retrovirus.

11. The viral vector system of claim 10, wherein said retrovirus is a lentivirus.

12. The viral vector system of claim 11, wherein said lentivirus is a human immunodeficiency virus or a simian immunodeficiency virus.

13. The viral vector system of claim 12, wherein said human immunodeficiency virus is HIV-1 or HIV-2.

14. The viral vector system of claim 5, wherein said fourth nucleic acid segment further comprises a gene encoding a marker protein selected from the group consisting of β-gal, fluorescence proteins, and luciferase.

15. The viral vector system of claim 1 further comprising promoters operatively linked to at least one of said first, said second, or said third nucleic acid segments.

16. The viral vector system of claim 3 further comprising promoters operatively linked to at least one of said first, said second, said third or said fourth nucleic acid segments.

17. A viral vector system comprising:
a) at least a first nucleic acid segment comprising a nucleotide sequence encoding at least a functional portion of a Gag polypeptide, and said first nucleic acid segment does not encode at least one of a functional Reverse Transcriptase polypeptide and a functional Integrase polypeptide; and,
b) at least a second nucleic acid segment comprising at least one nucleotide sequence encoding a fusion protein selected from the group consisting of:
i) a functional portion of a Vpr or a Vpx polypeptide and a functional portion of a Reverse Transcriptase polypeptide; and,
ii) a functional portion of a Vpr or Vpx polypeptide and a functional portion of an Integrase polypeptide;
wherein said functional portion of the Vpr or the Vpx polypeptide is capable of providing for the incorporation of said fusion protein into a viral particle and said second nucleic acid segment does not encode a functional Gag polypeptide; and,
wherein said viral vector system produces an infectious viral particle.

18. The viral vector system of claim 17, wherein
said first nucleic acid segment does not encode the functional Reverse Transcriptase and the functional Integrase polypeptide;
said second nucleic acid segment comprises a nucleotide sequence encoding the fusion protein comprising the functional portion of the Vpr or the Vpx polypeptide and the functional Reverse Transcriptase polypeptide fused in frame to the functional Integrase polypeptide.

19. The viral vector system of claim 17, wherein
said first nucleic acid segment does not encode the functional Reverse Transcriptase polypeptide and the functional Integrase polypeptide;
said second nucleic acid segment comprises a nucleotide sequence encoding the fusion protein comprising the functional portion of the Vpr or the Vpx polypeptide and the functional portion of the Reverse Transcriptase polypeptide; and,
said viral vector system further comprises a third nucleic acid segment comprising a nucleotide sequence encoding a second fusion protein comprising a second functional portion of the Vpr or the Vpx polypeptide and the functional portion of the Integrase polypeptide, wherein said third nucleic acid segment does not encode the functional Gag polypeptide.

20. The viral vector system of claim 17, further comprising at least a third nucleic acid segment comprising a nucleic acid sequence encoding a functional portion of an envelope polypeptide, wherein said third nucleic acid segment does not encode a functional Gag-Pol precursor polypeptide.

21. The viral vector system of claim 19, further comprising at least a fourth nucleic acid segment comprising a nucleic acid sequence encoding a functional portion of an envelope polypeptide, wherein said fourth nucleic acid segment does not encode a functional Gag-Pol precursor polypeptide.

22. The viral vector system of claim 17, wherein said first nucleic acid segment further comprises a Protease polypeptide.

23. The viral vector system of claim 20 further comprising at least a fourth nucleic acid segment comprising a nucleic acid sequence of interest, wherein said fourth nucleic acid segment does not encode a functional Gag-Pol precursor polypeptide wherein said viral vector system produces an infectious viral particle capable of transducing a target cell.

24. The viral vector system of claim 21 further comprising at least a fifth nucleic acid segment comprising a nucleic acid sequence of interest, wherein said fifth nucleic acid segment does not encode a functional Gag-Pol precursor polypeptide wherein said viral vector system produces an infectious viral particle capable of transducing a target cell.

25. The viral vector system of claim 23, wherein said nucleotide sequence of interest encodes a polypeptide.

26. The viral vector system of claim 25, wherein said polypeptide is a viral inhibitory polypeptide.

27. The viral vector system of claim 23, wherein said nucleotide sequence of interest is operably linked to a promoter active in a target cell.

28. The viral vector system of claim 17, wherein said functional portions of said Gag polypeptide, said Reverse Transcriptase polypeptide, and said Integrase polypeptide are from a retrovirus.

29. The viral vector system of claim 28, wherein said retrovirus is a lentivirus.

30. The viral vector system of claim 29, wherein said lentivirus is a human immunodeficiency virus or a simian immunodeficiency virus.

31. The viral vector system of claim 30, wherein said human immunodeficiency virus is HIV-1 or HIV-2.

32. The viral vector system of claim 23, wherein said fourth nucleic acid segment further comprises a gene encoding a marker protein selected from the group consisting of β-gal, fluorescence proteins, and luciferase.

33. The viral vector system of claim 17 further comprising promoters operatively linked to at least one of said first or said second nucleic acid segments.

34. The viral vector system of claim 19 further comprising promoters operably linked to at least one of said first, said second, or said third nucleic acid segment.

35. The viral vector system of claim 23 further comprising promoters operatively linked to at least one of said first, said second, said third or said fourth nucleic acid segments.
